SEQUENCE LISTING (32 sequences)

SEQ ID NO: 1 - protein, 51 aa, synthetic construct
GLAGGSQSQRAPDRVLCHSGQQQGLPRAAGGSVPHPRCHCGAGRADWPGPP

SEQ ID NO: 2 - protein, 15 aa, synthetic construct
ELDVCVEEAEGEAPW

SEQ ID NO: 3 - protein, 66 aa, synthetic construct
GLAGGSQSQRAPDRVLCHSGQQQGLPRAAGGSVPHPRCHCGAGRADWPGPPELDVCVEEAEGEAPW

SEQ ID NO: 4 - protein, 22 aa, synthetic construct
RAVSVNPELDVCVEEAEGEAPW

SEQ ID NO: 5 - protein, 73 aa, synthetic construct
RAVSVNPGLAGGSQSQRAPDRVLCHSGQQQGLPRAAGGSVPHPRCHCGAGRADWPGPPELDVCVEEAEGEAPW

SEQ ID NO: 6 - protein, 10 aa, synthetic construct
GFTFSDYGIA

SEQ ID NO: 7 - protein, 18 aa, synthetic construct
AFISDLAYTIYYADTVTG

SEQ ID NO: 8 - protein, 10 aa, synthetic construct
ARDNWDAMDY

SEQ ID NO: 9 - protein, 16 aa, synthetic construct
RSSQSLVHNNANTYLH

SEQ ID NO: 10 - protein, 7 aa, synthetic construct
KVSNRFS

SEQ ID NO: 11 - protein, 9 aa, synthetic construct
SQNTLVPWT

SEQ ID NO: 12 - protein, 113 aa, synthetic construct
DIQMTQSPSSLSASVGDRVTITCRSSQSLVHNNANTYLHWYQQKPGKAPKLLIYKVSNRFSGVPSRFSGSGSGTDFTLTISSLQPEDFATYYCSQNTLVPWTFGQGTKVEIKR

SEQ ID NO: 13 - protein, 120 aa, synthetic construct
EVQLVESGGGLVQPGGSLRLSCAASGFTFSDYGIAWVRQAPGKGLEWVAFISDLAYTIYYADTVTGRFTISRDNSKNTLYLQMNSLRAEDTAVYYCARDNWDAMDYWGQGTLVTVTVSSS

SEQ ID NO: 14 - protein, 248 aa, synthetic construct
DIQMTQSPSSLSASVGDRVTITCRSSQSLVHNNANTYLHWYQQKPGKAPKLLIYKVSNRFSGVPSRFSGSGSGTDFTLTISSLQPEDFATYYCSQNTLVPWTFGQGTKVEIKRGGGGSGGGGSGGGGSEVQLVESGGGLVQPGGSLRLSCAASGFTFSDYGIAWVRQAPGKGLEWVAFISDLAYTIYYADTVTGRFTISRDNSKNTLYLQMNSLRAEDTAVYYCARDNWDAMDYWGQGTLVTVTVSSS

SEQ ID NO: 15 - protein, 15 aa, synthetic construct
GGGGSGGGGSGGGGS

SEQ ID NO: 16 - protein, 70 aa, synthetic construct
TTTPAPRPPTPAPTIASQPLSLRPEACRPAAGGAVHTRGLDFACDIYIWAPLAGTCGVLLLSLVITLYCK

SEQ ID NO: 17 - protein, 40 aa, synthetic construct
RGRKKLLYIFKQPFMRPVQTTQEEDGCSCRFPEEEEGGCE

SEQ ID NO: 18 - protein, 113 aa, synthetic construct
LRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 21 aa, synthetic construct
MALPVTALLLPLALLLHAARP

SEQ ID NO: 20 - protein, 519 aa, synthetic construct
MALPVTALLLPLALLLHAARPTREQKLISEEDLPTRDIQMTQSPSSLSASVGDRVTITCRSSQSLVHNNANTYLHWYQQKPGKAPKLLIYKVSNRFSGVPSRFSGSGSGTDFTLTISSLQPEDFATYYCSQNTLVPWTFGQGTKVEIKRGGGGSGGGGSGGGGSEVQLVESGGGLVQPGGSLRLSCAASGFTFSDYGIAWVRQAPGKGLEWVAFISDLAYTIYYADTVTGRFTISRDNSKNTLYLQMNSLRAEDTAVYYCARDNWDAMDYWGQGTLVTVTVSSSGRDYKDDDDKMHTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - DNA, 338 bp, synthetic construct
  source 1..338: mol_type = other DNA
gatattcagatgactcagtctccgtccagcctctccgctagcgttggggaccgagtcacaatcacttgtaggtcttctcagagcctggtccacaataatgcgaacacttacttgcactggtatcagcagaagcctggtaaggccccgaaacttcttatatacaaagtatcaaatcgcttcagcggggttccgtcacgcttttctggtagcggatctggtacagatttcaccctcaccattagctctctccagcctgaagatttcgccacatattactgtagtcagaacacattggttccttggacgttcgggcagggaacgaaggtcgaaataaagcg

SEQ ID NO: 22 - DNA, 360 bp, synthetic construct
  source 1..360: mol_type = other DNA
gaggttcaactggtggagagcgggggtggtttggttcagcctggtgggtccctgaggctgtcatgtgctgcatcaggcttcacgttctccgactatggcatagcatgggtgagacaagctcctggaaaaggtcttgagtgggtggcttttatttctgatttggcgtacaccatatactacgctgacacagttactgggcggttcactatttccagagacaatagtaagaacactctttatcttcaaatgaactcattgagggcagaagacaccgctgtatattattgcgcgagagacaactgggatgcaatggactactggggtcaaggaaccttggtgacggtgactgttagcagtagc

SEQ ID NO: 23 - DNA, 744 bp, synthetic construct
  source 1..744: mol_type = other DNA
gatattcagatgactcagtctccgtccagcctctccgctagcgttggggaccgagtcacaatcacttgtaggtcttctcagagcctggtccacaataatgcgaacacttacttgcactggtatcagcagaagcctggtaaggccccgaaacttcttatatacaaagtatcaaatcgcttcagcggggttccgtcacgcttttctggtagcggatctggtacagatttcaccctcaccattagctctctccagcctgaagatttcgccacatattactgtagtcagaacacattggttccttggacgttcgggcagggaacgaaggtcgaaataaagcgcggtggcggtggctcgggcggtggtgggtcgggtggcggcggatctgaggttcaactggtggagagcgggggtggtttggttcagcctggtgggtccctgaggctgtcatgtgctgcatcaggcttcacgttctccgactatggcatagcatgggtgagacaagctcctggaaaaggtcttgagtgggtggcttttatttctgatttggcgtacaccatatactacgctgacacagttactgggcggttcactatttccagagacaatagtaagaacactctttatcttcaaatgaactcattgagggcagaagacaccgctgtatattattgcgcgagagacaactgggatgcaatggactactggggtcaaggaaccttggtgacggtgactgttagcagtagc

SEQ ID NO: 24 - DNA, 45 bp, synthetic construct
  source 1..45: mol_type = other DNA
ggtggcggtggctcgggcggtggtgggtcgggtggcggcggatct

SEQ ID NO: 25 - DNA, 210 bp, synthetic construct
  source 1..210: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaa

SEQ ID NO: 26 - DNA, 120 bp, synthetic construct
  source 1..120: mol_type = other DNA
cggggcagaaagaaactcctgtatatattcaaacaaccatttatgagaccagtacaaactactcaagaggaagatggctgtagctgccgatttccagaagaagaagaaggaggatgtgaa

SEQ ID NO: 27 - DNA, 342 bp, synthetic construct
  source 1..342: mol_type = other DNA
ctgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 28 - DNA, 45 bp, synthetic construct; identical to SEQ ID NO: 24
  source 1..45: mol_type = other DNA

SEQ ID NO: 29 - DNA, 1569 bp, synthetic construct
  source 1..1569: mol_type = other DNA
taagccgccatggccttaccagtgaccgccttgctcctgccgctggccttgctgctccacgccgccaggccgacgcgagagcaaaaacttatctctgaagaggacctcccaacgcgtgatattcagatgactcagtctccgtccagcctctccgctagcgttggggaccgagtcacaatcacttgtaggtcttctcagagcctggtccacaataatgcgaacacttacttgcactggtatcagcagaagcctggtaaggccccgaaacttcttatatacaaagtatcaaatcgcttcagcggggttccgtcacgcttttctggtagcggatctggtacagatttcaccctcaccattagctctctccagcctgaagatttcgccacatattactgtagtcagaacacattggttccttggacgttcgggcagggaacgaaggtcgaaataaagcgcggtggcggtggctcgggcggtggtgggtcgggtggcggcggatctgaggttcaactggtggagagcgggggtggtttggttcagcctggtgggtccctgaggctgtcatgtgctgcatcaggcttcacgttctccgactatggcatagcatgggtgagacaagctcctggaaaaggtcttgagtgggtggcttttatttctgatttggcgtacaccatatactacgctgacacagttactgggcggttcactatttccagagacaatagtaagaacactctttatcttcaaatgaactcattgagggcagaagacaccgctgtatattattgcgcgagagacaactgggatgcaatggactactggggtcaaggaaccttggtgacggtgactgttagcagtagcggccgcgactacaaagacgatgacgacaagatgcataccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 30 - DNA, 20 bp, synthetic construct
  source 1..20: mol_type = other DNA
ggtcctggaggtctagctct

SEQ ID NO: 31 - DNA, 20 bp, synthetic construct
  source 1..20: mol_type = other DNA
gcgaggagctggaagagctg

SEQ ID NO: 32 - DNA, 301 bp, synthetic construct
  source 1..301: mol_type = other DNA
ctgtcacctggagtctggggaagctaactggctggtcccaccccatcccagggctggctggcggctccgcgcagtcccagagggccccggatagggtgctctgccactccggacagcagcagggactgccgagagcagcaggaggctctgtcccccacccccgctgccactgtggagccgggagggctgactggccaggtcccccagagctggacgtgtgcgtggaggaggccgagggcgaggcgccgtggctgtggaccagtatttgtgtcttcatcaccctgttcctgctcagtgtgag

(19) United States
(12) Patent Application Publication
(10) Pub. No.: US 2025/0262303 A1
(43) Pub. Date: Aug. 21, 2025

(54) CHIMERIC ANTIGEN RECEPTOR T-CELLS TARGETING B CELLS EXPRESSING MEMBRANAL IGE

(71) Applicant: Ramot at Tel-Aviv University Ltd., Tel Aviv (IL)

(72) Inventors: Adi Barzel, Tel Aviv (IL); Tali Stauber, Tel Aviv (IL)

(21) Appl. No.: 19/054,302

(22) Filed: Feb. 14, 2025

Related U.S. Application Data
(60) Provisional application No. 63/554,255, filed on Feb. 16, 2024.

(51) Int. Cl. A61K 40/31; A61K 35/17; A61K 40/11; A61K 40/42; A61P 37/08; C07K 14/705; C07K 14/725; C07K 16/28; C12N 5/0783

(52) U.S. Cl. CPC A61K 40/31; A61K 35/17; A61K 40/11; A61K 40/421; A61P 37/08; C07K 14/7051; C07K 14/70517; C07K 14/70521; C07K 16/2803; C12N 5/0636; A61K 2239/13; A61K 2239/21; C07K 2317/565; C07K 2317/622; C12N 2510/00

(57) ABSTRACT

The present invention provides therapies for allergies. Specifically, the compositions of the present invention comprise engineered T cells comprising a chimeric antigen receptor that binds an IgE-BCR presented on B cells but not a secreted IgE. Therefore, the T cells eliminate IgE-producing B cells, prevent secretion of IgE and subsequently the symptoms of allergy.

### CROSS-REFERENCE TO RELATED APPLICATIONS

The present application claims priority to U.S. Provisional Patent Application No. 63/554,255, entitled “CHIMERIC ANTIGEN RECEPTOR T-CELLS TARGETING B CELLS EXPRESSING MEMBRANAL IGE”, filed on Feb. 16, 2024, the disclosure of which is hereby incorporated by reference in its entirety.

### SEQUENCE LISTING

The instant application contains a Sequence Listing which has been submitted electronically in xml format and is hereby incorporated by reference in its entirety. Said xml file, created on 2025 Feb. 10, is named RAMOT-0124 US Sequence listing.xml and is 34,074 bytes in size.

### FIELD OF THE INVENTION

The present invention is in the field of immunology and immunotherapy and relates to engineered immune cells expressing chimeric antigen receptors that bind the extracellular domain of IgE-B-Cell-Receptor (IgE-BCR) but do not bind secreted IgE.

### BACKGROUND OF THE INVENTION

The prevalence of IgE-mediated allergies has risen dramatically in the westernized world. The current treatments for allergies fail to offer a robust cure. In an attempt to eliminate all IgE-producing cells, use of chimeric antigen receptor (CAR) T cells targeting IgE was previously suggested (Lustgarten, J. & Eshhar, Z. Specific elimination of IgE production using T cell lines expressing chimeric T cell receptor genes. Eur. J. Immunol. 25, 2985-2991 (1995); Ward, D. E., Fay, B. L., Adejuwon, A., Han, H. & Ma, Z. Chimeric antigen receptors based on low-affinity mutants of FcϵRI Re-direct T cell specificity to cells expressing membrane IgE. Front. Immunol. 9, 1-11 (2018). However, these CARs target IgE epitopes found both on secreted IgE and on IgE B cell receptors (BCRs), which are membrane-anchored IgE antibodies. Subsequently, these previous anti-IgE CAR designs are prone to both activation and saturation by free IgE and in some cases also by FcεR bound IgE (FcERs are Fc receptors for IgE).

The monoclonal antibody (mAb) Quilizumab, developed by Genentech, specifically targets IgE-expressing B cells (Brightbill H D, Jeet S, Lin Z, Yan D, Zhou M, Tan M, Wu, Lawren C. et al. J. Clin. Invest. 2010, 120, 2218-2229, WO2008116149). Quilizumab targets an epitope on the M1′ domain, found only in the longer of two splice isoforms of IgE BCRs. In adults with inadequately controlled allergic asthma, Quilizumab reduced serum total and allergen-specific IgE by 30-40%, but had no impact on asthma exacerbations, lung function, or patient-reported symptom measures (Harris J M et al., Respir. Res. 2016, 17, 1-12).

Novel therapies for improved long-term control of allergies are still a highly desired goal.

### SUMMARY OF THE INVENTION

The present invention is based on our observation that CAR-T cells targeting the M1′ site of IgE-BCR that is expressed on the membranes of B cells successfully eliminated NALM6 cells expressing said IgE isomorph but not wild-type NALM6. Moreover, it was shown that the addition of free, secreted IgE did not reduce this effect. This targeted elimination was accompanied by an increase of gamma interferon production by the effector cells. Thus, and as detailed below, the main advantage of the present invention is that CAR T cells target only rare B cells and plasma cells that produce and secrete IgE, but are not influenced by secreted, freely circulating IgE. Therefore, the CAR T cells of the present invention are efficient in treating allergy and have fewer side effects common to CAR T cell treatment, such as cytokine storm.

According to one aspect, the present invention provides an immune cell engineered to express a chimeric antigen receptor (CAR) comprising an antigen binding domain (ABD) that specifically binds to an epitope within an amino acid sequence of an extracellular domain of a human IgE-BCR, wherein the amino acid sequence of the extracellular domain of the human IgE-BCR is absent in the sequence of a secreted IgE. According to some embodiments, the amino acid sequence of the extracellular domain of the human IgE-BCR is present only in the long isoform of IgE-BCR. According to other embodiments, the amino acid sequence is present only in the short isoform of IgE-BCR. According to further embodiments, the amino acid sequence is common to both the long and short isoforms of IgE-BCR. According to some embodiments, the amino acid sequence of the extracellular domain comprises a sequence selected from SEQ ID NO: 1, 2, 3, 4, and 5. According to some embodiments, the ABD comprises three complementarity-determining regions (CDR) of a light-chain variable domain (VL) having the amino acid sequence as set forth in SEQ ID NO: 12 and three CDR of a heavy-chain variable domain (VH) having the amino acid sequence as set forth in SEQ ID NO: 13. According to some embodiments, the ABD comprises a set of 6 complementarity-determining region (CDR) sequences, wherein the heavy chain CDR1 comprises the amino acid sequence SEQ ID NO: 6, the heavy chain CDR2 comprises the amino acid sequence SEQ ID NO: 7, the heavy chain CDR3 comprises the amino acid sequence SEQ ID NO: 8, the light chain CDR1 comprises the amino acid sequence SEQ ID NO: 9, the light chain CDR2 comprises the amino acid sequence SEQ ID NO: 10, and the light chain CDR3 comprises the amino acid sequence SEQ ID NO: 11. According to one embodiment, the ABD comprises a light-chain variable domain (VL) having the amino acid sequence as set forth in SEQ ID NO: 12 and a heavy-chain variable domain (VH) having the amino acid sequence as set forth in SEQ ID NO: 13. According to one embodiment, the ABD comprises a light-chain variable domain (VL) having an amino acid sequence having at least 85%, at least 90% or at least 95% sequence identity to SEQ ID NO: 12 and a heavy-chain variable domain (VH) having an amino acid sequence having at least 85%, at least 90% or at least 95% sequence identity to SEQ ID NO: 13, the ABD comprises 6 CDRs comprising the amino acid sequences SEQ ID NO: 6-11 and wherein there is no change in the CDRs sequences. According to some embodiments, the ABD is a single chain variable fragment (scFv) comprising the amino acid sequence SEQ ID NO: 14. According to some embodiments, the ABD is a single chain variable fragment (scFv) comprising an amino acid sequence having at least 85% sequence identity to SEQ ID NO: 14 and wherein the ABD comprises 6 CDRs comprising the amino acid sequences SEQ ID NO: 6-11 and wherein there is no change in the CDRs sequences. According to any one of the above embodiments, the CAR comprises a transmembrane domain (TM domain), a costimulatory domain, an activation domain, and a leading peptide. According to some embodiments, the TM domain comprises the amino acid sequence SEQ ID NO: 16, the costimulatory domain comprises the amino acid sequence SEQ ID NO: 17, the activation domain comprises the amino acid sequence SEQ ID NO: 18 and the leading peptide comprises the amino acid sequence SEQ ID NO: 19. According to any one of the above embodiments, the CAR is characterized by at least one of (i) TM domain comprises an amino acid sequence having at least 85%, at least 90%, or at least 95% sequence identity to amino acid sequence SEQ ID NO: 16, (ii) the costimulatory domain comprises an amino acid sequence having at least 85%, at least 90%, or at least 95% sequence identity to amino acid sequence SEQ ID NO: 17, (iii) the activation domain comprises an amino acid sequence having at least 85%, at least 90%, or at least 95% sequence identity to amino acid sequence SEQ ID NO: 18, and (iv) the leading peptide comprises an amino acid sequence having at least 85%, at least 90% or at least 95% sequence identity to amino acid sequence SEQ ID NO: 19. According to some embodiments, the CAR comprises or consists of amino acid sequence SEQ ID NO: 20. According to some embodiments, the CAR comprises an amino acid sequence having at least 85% sequence identity to amino acid sequence SEQ ID NO: 20 wherein the CAR comprises an ABD comprising 6 CDRs comprising the amino acid sequences SEQ ID NO: 6-11 and wherein there is no change in the CDRs sequences. According to any one of the embodiments of the application, the immune cell is selected from a T cell, a natural killer cell and a macrophage. According to some embodiments, the immune cell is a T cell such as CD4+ T cell and CD8+ T cell.

According to another aspect, the present invention provides a composition comprising a plurality of immune cells according to any one of the above aspects and embodiments, and a carrier. According to some embodiments, the carries is a pharmaceutically acceptable carrier and the composition is a pharmaceutical composition. Thus, according to yet another aspect, the present invention provides a pharmaceutical composition comprising a plurality of immune cells of any one of the above embodiments, and a pharmaceutically acceptable carrier and/or excipient. According to some embodiments, the pharmaceutical composition comprises a plurality of T cells comprising the CAR as described in the application. According to some embodiments, the T cells are selected from CD4+ T cells, CD8+ T cells, and combinations thereof. According to some embodiments, the pharmaceutical composition is for use in treating an allergy, allergic disease, condition or disorder.

According to yet another aspect, the present invention provides a chimeric antigen receptor (CAR) comprising an antigen binding domain (ABD) that specifically binds to an epitope within an amino acid sequence of an extracellular domain of a human IgE-BCR that is absent from a freely circulating IgE. According to some embodiments, the amino acid sequence of an extracellular domain of a human IgE-BCR is selected from SEQ ID NO: 1, 2, 3, 4, and 5. According to one embodiments, the extracellular domain comprises the amino acid sequence SEQ ID NO: 1. According to some embodiments, the present invention provides a chimeric antigen receptor (CAR) comprising an antigen binding domain (ABD) comprising three complementarity-determining regions (CDR) of a light-chain variable domain (VL) having the amino acid sequence as set forth in SEQ ID NO: 12 and three CDRs of a heavy-chain variable domain (VH) having the amino acid sequence as set forth in SEQ ID NO: 13. According to some embodiments, the ABD of the CAR comprises a set of 6 complementarity-determining region (CDR) sequences, wherein the heavy chain CDR1 comprises the amino acid sequence SEQ ID NO: 6, the heavy chain CDR2 comprises the amino acid sequence SEQ ID NO: 7, the heavy chain CDR3 comprises the amino acid sequence SEQ ID NO: 8, the light chain CDR1 comprises the amino acid sequence SEQ ID NO: 9, the light chain CDR2 comprises the amino acid sequence SEQ ID NO: 10, and the light chain CDR3 comprises the amino acid sequence

SEQ ID NO: 11. According to some examples, the ABD comprises a heavy-chain variable domain (VH) having the amino acid sequence as set forth in SEQ ID NO: 13 and a light-chain variable domain (VL) having the amino acid sequence as set forth in SEQ ID NO: 12. According to some examples, the ABD is a single chain variable fragment (scFv) comprising the amino acid sequence SEQ ID NO: 14. According to some examples, the ABD is a single chain variable fragment (scFv) comprising the amino acid sequence having at least 85% sequence identity to SEQ ID NO: 14 and wherein the ABD comprises 6 CDRs comprising the amino acid sequences SEQ ID NO: 6-11 and wherein there is no change in the CDRs sequences. According to some examples, the CAR comprises a transmembrane domain (TM domain), a costimulatory domain, an activation domain and a leading peptide. According to some examples, the TM domain comprises the amino acid sequence SEQ ID NO: 16, the costimulatory domain comprises the amino acid sequence SEQ ID NO: 17, the activation domain comprises the amino acid sequence SEQ ID NO: 18 and the leading peptide comprises the amino acid sequence SEQ ID NO: 19. According to some examples, the CAR is characterized by at least one of (i) TM domain comprises an amino acid sequence having at least 85%, at least 90%, or at least 95% sequence identity to amino acid sequence SEQ ID NO: 16, (ii) the costimulatory domain comprises an amino acid sequence having at least 85%, at least 90%, or at least 95% sequence identity to amino acid sequence SEQ ID NO: 17, (iii) the activation domain comprises an amino acid sequence having at least 85%, at least 90%, or at least 95% sequence identity to amino acid sequence SEQ ID NO: 18, and (iv) the leading peptide comprises an amino acid sequence having at least 85%, at least 90% or at least 95% sequence identity to amino acid sequence SEQ ID NO: 19. According to one example, the CAR comprises the amino acid sequence SEQ ID NO: 20. According to some embodiments, the CAR comprises an amino acid sequence having at least 85% sequence identity to amino acid sequence SEQ ID NO: 20 wherein the CAR comprises an ABD comprising 6 CDRs comprising the amino acid sequences SEQ ID NOs: 6-11 and wherein there is no change in the CDRs sequences.

According to one aspect, the present invention provides a nucleic acid molecule encoding the CAR according to any one of the embodiments of the present invention. According to some embodiments, the nucleic acid molecule comprises the nucleic acid sequence selected from SEQ ID NOs: 21, 22, 23, 25, 26, 27, 28 and a combination thereof. According to some embodiments, the nucleic acid molecule comprises sequence SEQ ID NO: 29.

According to another aspect, the present invention provides a nucleic acid construct comprising the nucleic acid molecule as described herein, operably linked to a promoter.

According to a further aspect, the present invention provides a vector comprising the nucleic acid molecule or the nucleic acid construct as described herein.

According to one aspect, the present invention provides a cell comprising the CAR, the nucleic acid molecule, the nucleic acid construct, or the vector as described herein. According to some examples, the cell expresses or is capable of expressing the CAR as described herein. According to some examples, the cell is an immune cell, such as a T cell, a natural killer cell and a macrophage.

According to yet another aspect, the present invention provides a method for treating an allergy in a subject in need thereof comprising administering to said subject a therapeutically effective amount of immune cells as described herein or a pharmaceutical composition comprising the immune cells.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In all figures, the statistical analysis performed is student t-test paired with following notifications: ns=non-significant, *p<0.05, **p<0.01, ***p<0.001, ****p<0.0001.

FIG. 1A shows an illustration of the IgE isoforms. From left to right, long isoform of IgE B cell receptor; short isoform of IgE B cell receptor (lacking the M1′ domain); secreted free IgE.

FIG. 1B shows the Q-CAR vector scheme.

FIG. 2A shows a schematic representation of the “IgE-BCR surrogate construct” for the short isoform lacking M1′ that was transduced into a NALM6 cell line (NALM6-S).

FIG. 2B shows a schematic representation of the “IgE-BCR surrogate construct” using the long-IgE-BCR isoform that was transduced into a NALM6 cell line (NALM6-L).

FIG. 3 shows the design for a transgenic mouse “knock-out mouse Extra Membranal Proximal Domain (EMPD) Knock-in human EMPD”, adopted from Vigl et al. 2017. Mice were designed with homozygous replacement of the endogenous murine EMPD by the human EMPD, by use of the CRISPR/Cas9 system.

FIG. 4A shows a histogram of the transduction efficiency of human lymphocytes determined by flow cytometry detecting mCherry fluorescence in non-transduced vs. transduced with Q-CAR.

FIG. 4B Fluorescence microscope imaging showing mCherry expression in transduced human lymphocytes.

FIG. 5 shows that the detection of mCherry by flow cytometry correlates with anti-FLAG staining (top) and anti-Myc staining (bottom). On the left, untransduced human lymphocytes; on right, human lymphocytes transduced with Q-CAR.

FIG. 6 shows activation of primary human anti-IgE-BCR-CAR-T cells by human cells expressing the M1′ segment as evident by a significant increase in IFN-gamma production (HEK293 and Daudi cell lines engineered to express M1′) but no activation by control cell lines not expressing M1′, nor IgE-secreting myeloma cell line U266. WO refers to untransduced T cells.

FIG. 7A shows a flow cytometry analysis of the CD69 activation marker expressed by regular Jurkat cells upon co-culture with either regular HEK293 cells (left) or HEK293 cells expressing the M1′ segment (right).

FIG. 7B shows flow cytometry analysis of the CD69 activation marker expressed by Jurkat cells transduced with anti-IgE-BCR CAR upon co-culture with either regular HEK293 cells (left) or the HEK293 cells expressing the M′ segment (right).

FIG. 7C shows data of FIG. 7A and FIG. 7B represented in a bar-graph.

FIG. 7D shows flow cytometry analysis of the CD69 activation marker expressed by regular Jurkat cells upon co-culture with either regular Daudi cells (left) or Daudi cells expressing the M1′ segment (right).

FIG. 7E shows flow cytometry analysis of the CD69 activation marker expressed by Jurkat cells transduced with anti-IgE-BCR CAR upon co-culture with either regular Daudi cells (left) or Daudi cells expressing the M1′ segment (right).

FIG. 7F shows the data of FIG. 7D and FIG. 7E represented in a bar-graph.

FIG. 8 shows the results of Luciferase killing assay, assessing the number of remaining target cells (that were genetically engineered to express luciferase firefly enzyme). The target cells—i.e., NALM6-L (here “NALM6-IgE”) were co-cultured for 13 hours with Q-CAR T cells, Effector to Target ratio 4:1. Control experiments were performed with non-transduced T cells and with NALM6 cells not expressing the M1′ domain. n=3.

FIG. 9 shows a flowcytometric analysis of a co-culture experiment of anti-IgE-BCR-CAR-T cells (“Q”) with a mixed population of NALM6/NALM6-L, showing the prominent elimination of the IgE-BCR+ population among the mixed targets population after 20 hours of co-culture.

FIG. 10 shows IFN gamma production by effector T cells or Q-CAR-T cells upon 8 hours co-culture with target-cells populations, showing non-activation of non-transduced human T lymphocytes (“T”), in contrast to Q-CAR-T cells (“Q”) that were activated upon co-culture with target cells with the long IgE-BCR-surrogate-construct (“NALM6-IgE”) as compared with co-culture with NALM6. The E:T ratio was 1:2, the CAR-T transduction rate was approximately 70%, and the co-culture duration was 8 hours; n=3.

FIG. 11 shows IFN-gamma production (ELISA) in the supernatant of co-culture experiments, with and without human IgE 5 μg/mL in medium, demonstrating specific activation only upon co-culture with Q-CAR T cells with NALM6-L target cell line, (here “NALM6-IgE”) that is not affected by the presence of free IgE.

FIG. 12A shows the “O-CAR” (O) vector design. “O” refers to a CAR construct adopted from Omalizumab (the variable domains of a therapeutic anti IgE antibody). The vector is identical to “Q-CAR” vector, except for the ectodomain.

FIG. 12B shows the “F-CAR” (F) vector design. “F” refers to a CAR construct using a mutated FcϵRIα IgE-Receptor. The vector is identical to the “Q-CAR” vector, except for the ectodomain.

FIG. 13A shows flow cytometric analysis of Q-CAR (Q), O-CAR (O), and F-CAR (F) anti-IgE CAR-T cells after 15 minutes incubation with free human IgE of 5 μg/mL and staining with anti-CD3 and anti-IgE; FIG. 13B shows a graphic representation of FIG. 13A as the mean percent of IgE-engaged-CARs from four different results.

FIG. 14 shows the effect of free IgE supplement 5 μg/mL to the culture media on IFN-gamma production by untransduced T-lymphocytes (T), Q-CAR (Q), O-CAR (O), or F-CAR (F). While O and F CAR-T cells are activated by the sole addition of free IgE to the media, Q-CAR T cells are not affected.

FIG. 15 shows the results of the Luciferase killing assay, which assessed the number of remaining target cells (that were genetically engineered to express luciferase firefly enzyme) at the end of co-culture. The target cells—i.e., NALM6-L (here “NALM6-IgE”) were co-cultured for 13 hours with Q-CAR T cells, Effector to Target ratio 4:1. Control experiments were held with O-CAR T cells, F-CAR T cells and non-transduced T cells, and with NALM6 not expressing the M1′ domain. n=3. All three Anti-IgE CAR-T cells (Q, O and F) totally eliminated NALM6-IgE cells, while the killing of NALM6 that do not express IgE was similar to non-transduced T cells.

FIG. 16 shows the effect of free IgE supplemented to the medium on killing by anti-IgE CAR-T cells. Shown herein are the results of the Luciferase killing assay, which assessed the remaining number of target cells (that were genetically engineered to express luciferase firefly enzyme) at the end of co-culture. The target cells NALM6-L (here “NALM6-IgE”) were co-cultured for 13 hours with Q-CAR T cells, with or without 5 μg/mL human IgE supplemented to the culture media. Effector to Target ratio 4:1. Control experiments were performed with O-CAR T cells, F-CAR T cells and non-transduced T cells, n=3. While killing by O and F CAR-T cells was decreased upon IgE supplementation to the media, killing by Q-CAR-T cells was not affected.

FIG. 17 shows the results of the LAD2 Mast cell degranulation assay: LAD2 Mast cells were pre-incubated with human IgE 1 μg/ml and then co-cultured with activated T cells or Q-CAR T cells (1:1 ratio, 50% CAR transduction rate among Q-CAR T cell population). The hexosaminidase-β activity was determined using PNAG as substrate, and the percentage of degranulation was calculated. Data is presented as mean±SD (3 different donors).

FIG. 18A shows ELISpot plates depicting IgE secreting cells (upper) and IgG secreting cells (lower) in the end of co-culture (72 h) of primary human B lymphocytes with same donor's T cells (T) or Q-CAR T cells (Q CAR) (T:B 5:1).

FIG. 18B shows number of IgE (left panel) and IgG (right panel) immunoglobulin-secreting cells as depicted by ELISpot plates of FIG. 18A, represented as the mean of three different donors.

FIG. 18C shows ELISA measurement of IgE (left) and IgG (right) in the supernatant after co-culture of primary human B cells for 48 h alone (-), with the same donor's T cells (T) or Q-CAR T cells (Q CAR) (T:B 1:2, n=3). Statistics: ns=non-significant, *p<0.05, **p<0.01, ***p<0.001, ****p<0.0001.

FIG. 19A-FIG. 19D show details of the experiment showing that Q-CAR T cells eliminate NALM6-L (luciferase+) cells in vivo. FIG. 19A shows the time line of the experiment. FIG. 19B shows IVIS photos following tumor expansion of the three treatment groups (1-second exposure except for the first image). FIG. 19C shows bioluminescence measurements (in one-minute exposure) curves over time demonstrating tumor expansion control by Q-CAR T cells in vivo. FIG. 19D shows Kaplan-Meier survival curves of three different treatment groups showing increased survival for mice treated with Q-CAR T cells.

### DETAILED DESCRIPTION OF THE INVENTION

Unless otherwise defined, all technical and/or scientific terms used herein have the same meaning as commonly understood by one of ordinary skill in the art to which the invention pertains. In case of conflict, the patent specification, including definitions, will control.

As discussed in the background, previous attempts to eliminate IgE-producing B cells by CAR T cell therapy did not lead to a therapeutic product. The approach in which CAR T-cells target all IgEs and do not discriminate between free circulating IgE and IgE-BCR (membrane-bound IgEs, mIgE, i.e., IgE bound to the membrane of B cells), is not feasible due to several practical considerations. Free IgE is abundant in the blood of allergic patients, much more than mIgE. Thus, free IgE bind and subsequently neutralize most of the CAR T cells that target domains shared by both free and membrane-bound IgE. Consequently, the CAR T-cells do bind and eliminate IgE-producing B cells. Another possible complication is the overactivation of CAR T cells as a result of non-specific binding of CAR T cells to free IgE due to the abundance of the latter, which may lead to a life-threatening reaction of the immune system, such as a cytokine storm.

The present application provides T cells comprising a chimeric antigen receptor designed to target a fragment of IgE that exists in membrane-anchored IgE but does not exist in free IgE—the M1′-domain. As shown in the Examples, CARs were expressed on human primary lymphocytes via retroviral transduction. The potency of the therapy of the T cells was tested on engineered NALM6 (Human B cell precursor leukemia) cell line expressing IgE-BCR-like surrogate constructs and showed outstanding effect on killing B cells presenting IgE on their membrane on one hand and lack of interactions with free IgE on the other hand.

IgE-BCR-targeting CAR-T cells successfully eliminated NALM6 cells expressing IgE-BCR, which is typically expressed on IgE-producing B cells, but not the wild-type NALM6 lacking IgE. This targeted elimination was accompanied by an increase in gamma interferon production by the effector cells. Considering that the M1′ domain does not exist in the freely circulating IgE present in serum, such IgE does not affect or compete on the binding of the CAR with the IgE bound/anchored to B-cells. It was shown that free IgE did not affect the efficacy of binding of the CAR T cell of the present invention to B-cells-presenting IgE.

According to the teachings of the present invention, engineered T cells comprising CAR that specifically bind domains of an IgE-BCR that do not present in free (secreted/circulating) IgE eliminate only B cells presenting such IgE and do not interact with free IgE. In this way, the therapy eliminates the IgE-producing B cells and is not affected by free IgE and consequently, does not cause overactivation of T cells.

Importantly, immunodeficient mice with tumors that resulted from the injection of NALM6 cells expressing IgE-BCR lived significantly longer after treatment with the CAR T cells of the present invention (Q-CAR T cells, see Examples section) than the mice that received untransduced T cells or were untreated. Thus, it is shown herein that Q-CAR successfully and specifically targets the M1′ domain of IgE-BCR in vivo and eliminates the B cells comprising BCR-IgE.

According to one aspect, the present invention provides an immune cell engineered to express a chimeric antigen receptor (CAR) comprising an antigen binding domain (ABD) that specifically binds to an epitope within an amino acid sequence of an extracellular domain of a human IgE-BCR, wherein the epitope and/or the amino acid sequence is absent in the secreted, freely circulating or receptor-bound, IgE. According to some embodiments, the terms “secreted” and “freely circulating”, when referring to IgE, may be used interchangeably. In some embodiments, the secreted IgE may be bound to a receptor and the terms “secreted IgE” and “receptor-bound IgE” may be used interchangeably. According to the teachings of the present invention, the immune cells comprising the CAR of the present invention do not specifically bind to such secreted, freely circulating or receptor-bound IgE.

As used herein, the term “IgE-B-Cell-Receptor (IgE-BCR)”, “membranal IgE” and “mIgE” may be used interchangeably and refer to IgE that is anchored to the surface of IgE-producing B cells. According to the principles of the present invention, the IgE-BCR-targeting CAR T may target any amino acid sequence that is specific to membrane-anchored IgE that is not present in freely circulating and/or receptor-bound IgE. According to some embodiments, the IgE-BCR-targeting CAR-T targets the M1′ domain of IgE-BCR. Therefore, according to some embodiments, the present invention provides an immune cell engineered to express a chimeric antigen receptor (CAR) comprising an antigen binding domain (ABD) that specifically binds to an epitope within a sequence of an extracellular domain of a human IgE-BCR that is absent from a freely circulating IgE.

The terms “absent in” and “devoid” when referring to IgE, refer to a section, domain or amino acid sequence that is not present, included or contained in a specific isoform, conformation or subtype of an IgE. Specifically, the term refers to a section, domain or sequence that is not present in secreted, freely circulating or receptor-bound IgE.

The terms “IgE” and “immunoglobulin E” are used herein interchangeably and refer to immunoglobulin E as well known in the art. Two types of IgE are referred: IgE that are anchored to the membrane of immune cells (IgE-BCR) that produce/secrete the IgE (i.e. B cells) and free IgE that are secreted by the cells. Secreted IgE include IgE that is secreted and receptor-bound IgE.

Two isoforms of human IgE-BCR exist: long and short. Both these isoforms comprise a domain denoted as M1 which is not present in free-circulating IgEs. The M1 domain of the long isoform comprises an insertion of 52 amino acids denoted M1′ (SEQ ID NO: 1). The long and the short isoforms comprise a common sequence SEQ ID NO: 2. These isoforms generate additional epitopes that do not present in freely circulating IgE, including several amino acids of the region of the C-terminus of the CH4 domain.

As used herein, the term “epitope” or “antigenic determinant” refers to a determinant or site on an antigen to which an antigen-binding domain of an antibody, or of a part of the antibody comprising said antigen-binding domain, specifically binds. In certain embodiments, the epitope includes chemically active surface groupings of molecules such as amino acids, sugar side chains, phosphoryl, or sulfonyl, and, in certain embodiments, may have specific three-dimensional structural characteristics, and/or specific charge characteristics. The epitopes of protein antigens can be demarcated into “linear epitopes” and “conformational epitopes”. As used herein, the term “linear epitope” refers to an epitope formed from a contiguous, linear sequence of linked amino acids. In some embodiments, an epitope is non-linear and the term “epitope” refers to non-linear epitope. An epitope typically includes at least 3, 4, 5, 6, 7, 8, 9, 10, 11, 12, 13, 14 or 15 amino acids in a unique spatial conformation. In some embodiments, an epitope includes fewer than 25, 24, 23, 22, 21, 20, 19, 18, 17, 16, 15, 14, 13, 12, 11, 10, 9, 8, 7, 6 or 5 amino acids in a unique spatial conformation. Generally, an antibody, or antigen-binding fragment thereof, specific for a particular target molecule will preferentially recognize and bind to a specific epitope on the target molecule within a complex mixture of proteins and/or macromolecules.

According to some embodiments, the CAR comprises an ABD that specifically binds to an epitope within the amino acid sequence that is present only in the long isoform of IgE-BCR. According to some embodiments, the CAR comprises an ABD that specifically binds to an epitope within the amino acid sequence of M1′-domain. According to some embodiments, the CAR comprises an ABD that specifically binds to an epitope within the amino acid sequence SEQ ID NO: 1.

According to some embodiments, the CAR comprises an ABD that specifically binds to an epitope within the amino acid sequence that is common to long and short isoforms of a IgE-BCR. According to some embodiments, the CAR comprises an ABD that specifically binds to an epitope within the amino acid sequence SEQ ID NO: 2.

According to some embodiments, the CAR comprises an ABD that specifically binds to an epitope within the amino acid sequence SEQ ID NO: 3.

According to some embodiments, the CAR comprises an ABD that specifically binds to an epitope within the amino acid sequence that is present only in the short isoform of IgE-BCR. According to some embodiments, the CAR comprises an ABD that specifically binds to an epitope within the amino acid sequence SEQ ID NO: 4.

According to some embodiments, the CAR comprises an ABD that specifically binds to an epitope within the amino acid sequence SEQ ID NO: 5.

According to some embodiments, the CAR comprises an ABD that specifically binds to an epitope within the amino acid sequence selected from SEQ ID NOs: 1, 2, 3, 4 and 5.

The terms “immune cells” and “immunological cells” are used herein interchangeably and refer to cells that are involved in an immune response, e.g., the promotion of an immune response. According to some embodiments, the immune cells are selected from T cells, natural killer (NK) cells, and myeloid-derived phagocytes. According to some embodiments, the term refers to cells of the immune system that are commonly used in immunotherapy such as T cells, natural killer (NK) cells, macrophages and tumor-infiltrating lymphocytes (TIL). The term “T cell” as used herein refers to a thymus-derived lymphocyte that participates in a variety of cell-mediated immune reactions, as well known in the art. The term “T cell” refers to a type of white blood cell that can be distinguished from other white blood cells by the presence of a T cell receptor on the cell surface. There are several subsets of T cells, including, but not limited to, T helper cells (a.k.a. Tx cells or CD4+ T cells) and subtypes, including T_{H}1, T_{H}2, T_{H}3, T_{H}17, T_{H}9, and T_{FH} cells, cytotoxic T cells (i.e., Tc cells, CD8+ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. Treg cells or suppressor T cells) and subtypes, including CD4⁺FOXP3⁺ Treg cells, CD4⁺FOXP3⁻ T_{reg} cells, Tr1 cells, Th3 cells, and T_{reg}17 cells, natural killer T cells (a.k.a. NKT cells), mucosal-associated invariant T cells (MAITs), and gamma delta T cells (γδ T cells), including Vγ9/Vδ2 T cells. Any one or more of the aforementioned or unmentioned T cells may be employed for a method of use of the invention. According to any one of the above embodiments, the T cell is selected from a CD4+ T-cell and a CD8+ T-cell. According to some embodiments, the immune cell is selected from a T cell, a natural killer cell and a macrophage. According to some embodiments, the immune cell is a T cell.

As used herein, the term “engineered immune cell” refers to an immune cell, also referred to as an “immune effector cell”, that has been genetically modified by the addition of extra genetic material in the form of DNA or RNA to the total genetic material of the cell. According to some embodiments herein, the engineered immune cells have been genetically modified to express a chimeric antigen receptor.

In one embodiment, the term “engineered to express” refers to cells that are modified to express a polypeptide, protein or proteins, which are not natively expressed in those cells. In one embodiment, such expression may be as a result of the integration of a sequence of interest within a genome of the cell, to facilitate such expression, or, in another embodiment, a specific mutation of a sequence of interest in the genome, or in another embodiment, may be a result of extrachromosomal expression, such as via a plasmid, as will be appreciated by one skilled in the art. The term refers both to cell that constitutively express the polypeptide, protein or proteins or is capable of expressing it upon a stimulus, e.g., under specific conditions.

The terms “chimeric antigen receptor” and “CAR” are used herein interchangeably and refer to an engineered recombinant polypeptide or receptor which are grafted onto cells and comprising at least (1) an extracellular domain (“ectodomain”) comprising an antigen-binding region, e.g., a single chain variable fragment of an antibody or a whole antibody, (2) a transmembrane domain to anchor the CAR into a cell, and (3) one or more cytoplasmic signaling domains (also referred to herein as “an intracellular signaling domains”). The extracellular domain comprises an antigen binding domain (ABD) and, optionally, a spacer or hinge region. The extracellular domain also comprises a leading peptide that targets the CAR to the extracellular region. The antigen-binding domain of a CAR targets a specific antigen. The targeting regions may comprise full length heavy chains, Fab fragments, or single-chain variable fragments (scFvs). The antigen binding domain can be derived from the same species or a different species in which the CAR will be used. In one embodiment, the antigen binding domain is an scFv. It is clear that in case the ABD comprises more than one polypeptide, more than one scFv may be used and therefore the ABD may comprise more than one Fv.

The extracellular spacer or hinge region of a CAR is located between the antigen-binding domain and the transmembrane domain. Extracellular spacer domains may include, but are not limited to, Fc fragments of antibodies or fragments or derivatives thereof, hinge regions of antibodies or fragments or derivatives thereof, constant domains such as CH2 regions or CH3 regions of antibodies, accessory proteins, artificial spacer sequences or combinations thereof.

The term “transmembrane domain” refers to the region of the CAR, which crosses or bridges the plasma membrane. The transmembrane domain of the CAR of the invention is the transmembrane region of a transmembrane protein, an artificial hydrophobic sequence or a combination thereof.

The term “intracellular domain” refers to the intracellular part of the CAR and may be an intracellular domain of the T cell receptor or of any other receptor (e.g., TNFR superfamily member) or portion thereof, such as an intracellular activation domain (e.g., an immunoreceptor tyrosine-based activation motif (ITAM)-containing T cell activating motif), one or more intracellular costimulatory domain, or both.

The term “antigen-binding portion”, “antigen-binding region” and “antigen-binding domain” are used herein interchangeably and refer to one or more fragments of an antibody that retain the ability to specifically bind to an antigen. It has been shown that the antigen-binding function of an antibody can be performed by fragments of a full-length antibody. Such antibody embodiments may also comprise bispecific, dual-specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody; (v) a dAb, which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). Such single-chain antibodies are also intended to be encompassed within the term “antigen-binding portion” of an antibody. In certain embodiments of the invention, scFv molecules are incorporated into a fusion protein. According to some embodiments, the antigen binding domain is a scFv.

The terms “binds specifically” or “specific for” with respect to an antigen-binding domain of an antibody, of a fragment thereof or of a CAR refers to an antigen-binding domain that recognizes and binds to a specific antigen, but does not substantially recognize or bind other molecules in a sample. The term encompasses that the antigen-binding domain binds to its antigen with high affinity and binds to other antigens with low affinity. An antigen-binding domain that binds specifically to an antigen from one species may bind also to the same antigen from another species. This cross-species reactivity is not contrary to the definition of that antigen-binding domain as specific. An antigen-binding domain that specifically binds to an antigen may bind also to different allelic forms of the antigen (allelic variants, splice variants, isoforms etc.). This cross-reactivity is not contrary to the definition of that antigen-binding domain as specific.

According to some embodiments, the ABD that specifically binds to amino acid sequence SEQ ID NO: 1 comprises 6 complementarity-determining region (CDR) sequences, wherein 3 CDRs of a light-chain variable domain (VL) are CDRs present in the sequence SEQ ID NO: 12, and 3 CDRs of a heavy-chain variable domain (VH) are CDRs present in the sequence SEQ ID NO: 13. According to one embodiment, the ABD comprises a set of 6 complementarity-determining region (CDR) sequences, wherein the heavy chain CDR1 comprises the amino acid sequence SEQ ID NO: 6, the heavy chain CDR2 comprises the amino acid sequence SEQ ID NO: 7, the heavy chain CDR3 comprises the amino acid sequence SEQ ID NO: 8, the light chain CDR1 comprises the amino acid sequence SEQ ID NO: 9, the light chain CDR2 comprises the amino acid sequence SEQ ID NO: 10, and the light chain CDR3 comprises the amino acid sequence SEQ ID NO: 11. According to some embodiments, the ABD comprises a VH domain and VL domain, wherein the CDRs of the VH and VL domains form a pharmacophore formed by CDRs present in SEQ ID NOs: 12 and 13. The term “pharmacophore” generally refers to the commonly used IUPAC definition, i.e. “an ensemble of steric and electronic features that is necessary to ensure the optimal supramolecular interactions with a specific biological target and to trigger (or block) its biological response”. As used herein, the term “CDR” refers to the complementarity determining region within antibody variable sequences. There are three CDRs in each of the variable regions of the heavy chain (HC) and the light chain (LC), which are designated CDR1, CDR2 and CDR3 (or specifically HC CDR1, HC CDR2, HC CDR3, LC CDR1, LC CDR2, and LC CDR3), for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. According to other embodiments, the CDRs are according to IMGT. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262 (5): 732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

According to some embodiments, the ABD comprises a heavy-chain variable domain (VH) having the amino acid sequence as set forth in SEQ ID NO: 13 and a light-chain variable domain (VL) having the amino acid sequence as set forth in SEQ ID NO: 12. According to some embodiments, the VH and VL domains are linked by a linker to form a single chain variable fragment (scFv). According to some embodiments, the C terminal end of the VH domain and the N terminal end of the VL domain are linked by a linker to form a single chain variable fragment (scFv). According to some embodiments, the N terminal end of the VH domain and the C terminal end of the VL domain are linked by a linker to form a single chain variable fragment (scFv). According to some embodiments, the linker comprises the amino acid sequences SEQ ID NO: 15. Therefore, according to some embodiments, the ABD is a single chain variable fragment (scFv). According to some embodiments, the ABD is an scFv comprising the amino acid sequence SEQ ID NO: 14.

According to any one of the above embodiments, the CAR comprises a transmembrane domain (TM domain), a costimulatory domain, an activation domain and a leading peptide.

According to some embodiments, the TM domain is a TM domain of a receptor selected from CD28 and CD8. According to one embodiment, the TM domain is a TM domain of CD28. According to one embodiment, the TM domain comprises the amino acid sequence SEQ ID NO: 16.

According to any one of the above embodiments, the costimulatory domain is selected from a costimulatory domain of a protein selected from CD28, 4-1BB, OX40, iCOS, CD27, CD80, CD70, and a combination thereof. According to some embodiments, the costimulatory domain is a costimulatory domain of CD28. According to some embodiments, the costimulatory domain is a costimulatory domain of 4-1BB. According to some embodiments, the costimulatory domain comprises the amino acid sequence SEQ ID NO: 17. According to some embodiments, the costimulatory domain is a costimulatory domain of OX40. According to some embodiments, the CAR comprises a costimulatory domain of CD28 and of 4-1BB. According to some embodiments, the CAR comprises a costimulatory domain of CD28 and of OX40. According to some embodiments, the CAR comprises two costimulatory domains.

According to any one of the above embodiments, the TM domain and the costimulatory domain are both derived from CD28.

According to any one of the above embodiments, the antigen binding domain is linked to the TM domain via a spacer.

According to any one of the above embodiments, the activation domain is selected from a FcRγ and a CD3-ζ activation domains. According to some embodiments, the activation domain is a CD3-ζ activation domain. According to some embodiments, the activation domain comprises the amino acid sequence SEQ ID NO: 18.

According to some embodiments, the CAR comprises a leading peptide. The term “leader peptide”, “leading peptide”, “lead peptide”, “signaling peptide” and “signal peptide” are used herein interchangeably and refer to a peptide that translocates, signals, or prompts translocation of the target protein to the cellular membrane. According to one embodiment, the leading peptide comprises the amino acid sequence SEQ ID NO: 19.

According to some embodiments, the TM domain is a TM domain of a receptor selected from CD28a, the costimulatory domain is a costimulatory domain of 4-1BB and the activation is a CD3-ζ activation domain. According to one embodiment, the TM domain comprises the amino acid sequence SEQ ID NO: 16, the costimulatory domain comprises the amino acid sequence SEQ ID NO: 17, the activation domain comprises the amino acid sequence SEQ ID NO: 18 and the leading peptide comprises the amino acid sequence SEQ ID NO: 19. According to some embodiments, the CAR comprises the amino acid sequence SEQ ID NO: 20.

According to some embodiments, the present invention provides an immune cell engineered to express a chimeric antigen receptor (CAR) comprising an antigen binding domain (ABD) binds specifically to an amino acid sequence selected from SEQ ID NOs: 1, 2, 3, 4 and 5, a transmembrane domain (TM domain), a costimulatory domain, an activation domain and a leading peptide, wherein the TM domain comprises the amino acid sequence SEQ ID NO: 16, the costimulatory domain comprises the amino acid sequence SEQ ID NO: 17, the activation domain comprises the amino acid sequence SEQ ID NO: 18 and the leading peptide comprises the amino acid sequence SEQ ID NO: 19. According to some embodiments, the present invention provides an immune cell engineered to express a chimeric antigen receptor (CAR) comprising an antigen binding domain (ABD) binds specifically to the amino acid sequence SEQ ID NO: 1, a TM domain comprising the amino acid sequence SEQ ID NO: 16, a costimulatory domain comprising the amino acid sequence SEQ ID NO: 17, an activation domain comprising the amino acid sequence SEQ ID NO: 18 and a leading peptide comprising the amino acid sequence SEQ ID NO: 19. According to some embodiments, the ABD comprises a set of 6 complementarity-determining region (CDR) sequences, wherein the heavy chain CDR1 comprises the amino acid sequence SEQ ID NO: 6, the heavy chain CDR2 comprises the amino acid sequence SEQ ID NO: 7, the heavy chain CDR3 comprises the amino acid sequence SEQ ID NO: 8, the light chain CDR1 comprises the amino acid sequence SEQ ID NO:9, the light chain CDR2 comprises the amino acid sequence SEQ ID NO: 10, and the light chain CDR3 comprises the amino acid sequence SEQ ID NO: 11. According to some embodiments, the ABD comprises a VL having the amino acid sequence as set forth in SEQ ID NO: 12 and a VH having the amino acid sequence as set forth in SEQ ID NO: 13. According to some embodiments, the CAR comprises the amino acid sequence SEQ ID NO: 20. According to some embodiments, the CAR comprising the amino acid sequence SEQ ID NO: 20 is referred to as Q-CAR, as in Examples. Therefore, according to some embodiments, the present invention provides an immune cell engineered to express a CAR comprising the amino acid sequence SEQ ID NO: 20. According to some embodiments, the immune cell is a T cell.

According to any one of the aspects and embodiments of the invention, the terms “peptide comprising the amino acid sequence set forth in SEQ ID NO: X”, “peptide comprising SEQ ID NO: X” and “peptide having SEQ ID NO: X” are used herein interchangeably. The terms “peptide consisting of the amino acid sequence set forth in SEQ ID NO: X”, “peptide consisting of SEQ ID NO: X” and “peptide of SEQ ID NO: X” are used herein interchangeably.

The same rule holds for nucleic acid sequence. Thus, the terms “nucleic acid comprising the nucleic acid sequence set forth in SEQ ID NO: X”, “nucleic acid comprising SEQ ID NO: X” and “nucleic acid having SEQ ID NO: X” are used herein interchangeably. The terms “nucleic acid consisting of the nucleic acid sequence set forth in SEQ ID NO: X”, “nucleic acid consisting of SEQ ID NO: X” and “nucleic acid of SEQ ID NO: X” are used herein interchangeably.

In the present invention, a particular amino acid sequence encompasses also the analog of that sequence. The term “peptide analog”, “analog” and “sequence analog” are used herein interchangeably and refer to an analog of a peptide having at least 85% sequence identity with the original peptide, wherein the analog retains the activity and the specificity of the original peptide. Thus, the terms “analog”, “functional analog” and “active analog” are used interchangeably. The term “analog” refers to a peptide, polypeptide or protein that contains substitutions, rearrangements, deletions, additions and/or chemical modifications in the amino acid sequence of the parent peptide. According to some embodiments, the peptide analog has at least 80%, at least 90% or at least 95% sequence identity to the original peptide. According to one embodiment, the analog has about 70% to about 95%, about 80% to about 90% or about 85% to about 95% sequence identity to the original peptide. According to some embodiments, the analog of the present invention comprises the sequence of the original peptide in which 1, 2, 3, 4, or 5 substitutions were made. According to any one of the above embodiments, the change in the amino acid is not in the CDRs. Therefore, the analog of the present invention comprises the CDRs of the ABD.

The substitutions of the amino acids may be a conservative or a non-conservative substitution. The non-conservative substitution encompasses a substitution of one amino acid by any other amino acid. In one particular embodiment, the amino acid is substituted by a non-natural amino acid.

The term “conservative substitution” as used herein denotes the replacement of an amino acid residue by another, without altering the overall conformation and biological activity of the peptide, including, but not limited to, a replacement of an amino acid with one having similar properties (such as, for example, polarity, hydrogen bonding potential, acidic, basic, shape, hydrophobic, aromatic, and the like). Amino acids with similar properties are well-known in the art. For example, according to one table known in the art, the following six groups each contain amino acids that are conservative substitutions for one another: (1) Alanine (A), Serine(S), Threonine (T); (2) Aspartic acid (D), Glutamic acid E; (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W).

According to some embodiments, the present invention provides an immune cell comprising a CAR comprising an ABD comprising a heavy-chain variable domain (VH) having an amino acid sequence having at least 85% sequence identity to SEQ ID NO: 13 and a light-chain variable domain (VL) having an amino acid sequence having at least 85% sequence identity to SEQ ID NO: 12, wherein the ABD comprises 6 CDRs comprising the amino acid sequences SEQ ID NO: 6-11 and wherein there is no change in the CDRs sequences. According to some embodiments, the VH and VL domains are linked by a linker to form a single chain variable fragment (scFv). According to some embodiments, the linker comprises the amino acid sequences having at least 85% sequence identity to SEQ ID NO: 15. Therefore, according to some embodiments, the ABD is a single chain variable fragment (scFv). According to some embodiments, the ABD is an scFv comprising an amino acid sequence having at least 85% sequence identity to SEQ ID NO: 14, wherein the ABD comprises 6 CDRs comprising the amino acid sequences SEQ ID NO: 6-11 and wherein there is no mutation in the CDRs sequences. According to some embodiments, the CAR comprises a TM domain, a costimulatory domain comprising, and an activation domain, wherein the TM domain comprises an amino acid sequence having at least 85% sequence identity to SEQ ID NO: 16, and/or a costimulatory domain comprises an amino acid sequence having at least 85% sequence identity to SEQ ID NO: 17, and/or an activation domain comprises an amino acid sequence having at least 85% sequence identity to SEQ ID NO: 18 and/or a leading peptide comprises an amino acid sequence having at least 85% sequence identity to SEQ ID NO: 19. According to some embodiments, the CAR comprises an amino acid sequence having at least 85% sequence identity SEQ ID NO: 20, wherein the CAR comprises an ABD comprising 6 CDRs comprising the amino acid sequences SEQ ID NO: 6-11 and wherein there is no mutation in the CDRs sequences. Therefore, according to some embodiments, the present invention provides an immune cell engineered to express a CAR comprising an amino acid sequence having at least 85% sequence identity SEQ ID NO: 20, wherein the CAR comprises an ABD comprising 6 CDRs comprising the amino acid sequences SEQ ID NO: 6-11 and wherein there is no mutation in the CDRs sequences. According to some embodiments, the present invention provides an immune cell expressing a CAR comprising an amino acid sequence having at least 85% sequence identity SEQ ID NO: 20, wherein the CAR comprises an ABD comprising 6 CDRs comprising the amino acid sequences SEQ ID NO: 6-11 and wherein there is no change in the CDRs sequences. According to some embodiments, the sequence identity is at least 90% at least 92%, at least 95% at least 98% or at least 99%. According to some embodiments, the immune cell is a T cell.

According to any one of the above embodiments, the cell is engineered to express the CAR upon a stimulus.

According to any one of the above embodiments, the cell is engineered to co-express an immune system effector. According to some embodiments, the immune system effector is selected from a cytokine, a chemokine, an immune checkpoint molecule or an inhibitor.

According to any one of the above embodiments, the cell is engineered to co-express a safety switch allowing to inducibly eliminate CAR-expressing cells.

In some examples, the immune cells of the present invention encode a caspase gene fused to an inducible dimerization domain. The default is the expression of an inactive monomeric caspase. However, if an adverse outcome is seen and quick shutoff is desired, the addition of a small membrane-permeable molecule allows the dimerization of the caspase and the subsequent induction of apoptosis. Apoptosis induced by caspase dimerization is independent on cell cycling, it is quick, non-immunogenic and has little bystander effect compared to common alternatives for induced cell death. According to some embodiments, the construct encoding the CAR-Q as defined in the Examples includes a caspase gene fused to an inducible dimerization domain which can induce apoptosis upon addition of a small membrane permeable dimerization inducer molecule. According to some embodiments, the inducible dimerization domain is a modified human FK-binding protein. According to some embodiments, the small membrane permeable dimerization inducer molecule is AP1903.

Alternative methods of cell death induction rely on HSV TK, cytidine deaminase or diphtheria toxin receptor.

According to another aspect, the present invention provides a composition comprising a plurality of immune cells according to any one of the above aspects and embodiments, and a carrier. All terms, embodiments and definitions disclosed in any one of the above aspects apply and are encompassed herein as well.

The term “plurality” as used herein has a meaning of two or more such elements.

The term “carrier” as used herein refers to as a class any compound or composition useful in facilitating storage, stability, administration, cell targeting and/or delivery of the topical composition, including, without limitation, suitable vehicles, skin conditioning agents, skin protectants, diluents, emollients, solvents, excipients, pH modifiers, salts, colorants, rheology modifiers, thickeners, lubricants, humectants, antifoaming agents, erodible polymers, hydrogels, surfactants, emulsifiers, emulsion stabilizers, adjuvants, surfactants, preservatives, chelating agents, fatty acids, mono-di- and tri-glycerides and derivates thereof, waxes, oils and water. According to some embodiments, the composition is a pharmaceutical composition and the carrier is a pharmaceutically acceptable carrier.

Thus, according to some embodiments, the present invention provides a pharmaceutical composition comprising a plurality of the immune cells according to any one of the above aspects and embodiments, and a pharmaceutically acceptable carrier and/or excipient.

According to some embodiments, the present invention provides a pharmaceutical composition comprises a plurality of immune cell engineered to express a chimeric antigen receptor (CAR) that binds specifically to an epitope within an amino acid sequence of an extracellular domain of a human IgE-BCR that is absent from a freely circulating IgE. According to some embodiments, the ABD specifically binds to M1′-domain of a human immunoglobulin E (IgE)-B-cell receptor. According to some embodiments, the ABD specifically binds to an amino acid sequence selected from SEQ ID NO: 1, 2, 3, 4, and 5. According to some embodiments, the ABD specifically binds to the amino acid sequence SEQ ID NO: 1. According to some embodiments, the ABD comprises a VH having the amino acid sequence as set forth in SEQ ID NO: 13 and a VL having the amino acid sequence as set forth in SEQ ID NO: 12. According to some embodiments, the ABD is an scFv comprising the amino acid sequence SEQ ID NO: 14. According to some embodiments, the pharmaceutical composition comprises a plurality of immune cells engineered to express a CAR comprising the amino acid sequence SEQ ID NO: 20. According to some embodiments, the immune cells are selected from T cells, natural killer (NK) cells and macrophages. According to some embodiments, the immune cells are T cells. According to some embodiments, the T cells are selected from CD4+ T-cells, CD8+ T-cells and a combination thereof. According to some embodiments, the CAR comprises an ABD comprising a VH having an amino acid sequence having at least 85% sequence identity to SEQ ID NO: 13 and a VL having an amino acid sequence having at least 85% sequence identity to SEQ ID NO: 12, wherein the ABD comprises 6 CDRs comprising the amino acid sequences SEQ ID NO: 6-11 and wherein there is no mutation in the CDRs sequences. According to some embodiments, the ABD is an scFv comprising an amino acid sequence having at least 85% sequence identity to SEQ ID NO: 14, wherein the ABD comprises 6 CDRs comprising the amino acid sequences SEQ ID NO: 6-11 and wherein there is no amendment in the CDRs sequences. According to some embodiments, the CAR comprises a TM domain, a costimulatory domain comprising, and an activation domain, wherein the TM domain comprises an amino acid sequence having at least 85% sequence identity to SEQ ID NO: 16, and/or a costimulatory domain comprises an amino acid sequence having at least 85% sequence identity to SEQ ID NO: 17, and/or an activation domain comprises an amino acid sequence having at least 85% sequence identity to SEQ ID NO: 18 and/or a leading peptide comprises an amino acid sequence having at least 85% sequence identity to SEQ ID NO: 19. According to some embodiments, the pharmaceutical composition comprises a plurality of immune cells comprising and/or expressing and/or capable of expressing a CAR comprising an amino acid sequence having at least 85% sequence identity SEQ ID NO: 20, wherein the CAR comprises an ABD comprising 6 CDRs comprising the amino acid sequences SEQ ID NO: 6-11 and wherein there is no alteration in the CDRs sequences. According to some embodiments, the immune cell is a T cell. The term “pharmaceutical composition” as used herein refers to a composition comprising the engineered immune cells of the present invention formulated together with one or more pharmaceutically acceptable carriers.

The formulation of the pharmaceutical composition may be adjusted according to the application. In particular, the pharmaceutical composition may be formulated using a method known in the art so as to provide rapid, continuous or delayed release of the active ingredient after administration to mammals. For example, the formulation may be any one selected from among plasters, granules, lotions, liniments, lemonades, aromatic waters, powders, syrups, ophthalmic ointments, liquids and solutions, aerosols, extracts, elixirs, ointments, fluidextracts, emulsions, suspensions, decoctions, infusions, ophthalmic solutions, tablets, suppositories, injections, spirits, capsules, creams, troches, tinctures, pastes, pills, and soft or hard gelatin capsules.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well-known in the art. The compositions may contain other active compounds providing supplemental, additional, or enhanced therapeutic functions, e.g. solid carriers or excipients such as, for example, lactose, starch or talcum or liquid carriers such as, for example, water, fatty oils or liquid paraffins.

Other carriers or excipients which may be used include, but are not limited to, materials derived from animal or vegetable proteins, such as gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; polypeptide/protein or polysaccharide complexes such as gelatin-acacia complexes; sugars such as mannitol, dextrose, galactose and trehalose; cyclic sugars such as cyclodextrin; inorganic salts such as sodium phosphate, sodium chloride and aluminum silicates; and amino acids having from 2 to 12 carbon atoms and derivatives thereof such as, but not limited to, glycine, L-alanine, L-aspartic acid, L-glutamic acid, L-hydroxyproline, L-isoleucine, L-leucine and L-phenylalanine. Each possibility represents a separate embodiment of the present invention.

Solutions or suspensions used for parenteral, intradermal, or subcutaneous application typically include the following components: a sterile diluent such as water for injection, saline solution, fixed oils, polyethylene glycols, glycerin, propylene glycol (or other synthetic solvents), antibacterial agents (e.g., benzyl alcohol, methyl parabens), antioxidants (e.g., ascorbic acid, sodium bisulfite), chelating agents (e.g., ethylenediaminetetraacetic acid), buffers (e.g., acetates, citrates, phosphates), and agents that adjust tonicity (e.g., sodium chloride, dextrose). The pH can be adjusted with acids or bases, such as hydrochloric acid or sodium hydroxide, for example. The parenteral preparation can be enclosed in ampules, disposable syringes or multiple dose glass or plastic vials.

Pharmaceutical compositions adapted for parenteral administration include, but are not limited to, aqueous and non-aqueous sterile injectable solutions or suspensions, which can contain antioxidants, buffers, bacteriostats and solutes that render the compositions substantially isotonic with the blood of an intended recipient. Such compositions can also comprise water, alcohols, polyols, glycerin and vegetable oils, for example. Extemporaneous injection solutions and suspensions can be prepared from sterile powders, granules and tablets. Such compositions preferably comprise a therapeutically effective amount of a compound of the invention and/or other therapeutic agent(s), together with a suitable amount of carrier so as to provide the form for proper administration to the subject.

According to some embodiments, the pharmaceutical composition is formulated for a parenteral administration. According to some embodiments, the pharmaceutical composition is formulated for an administration n route selected from intravenous, subcutaneous, intracutaneous, intramuscular, intraarticular, intraarterial, intrasynovial, intrasternal, intrathecal, intralesional, intraperitoneal and intracranial injection. According to some embodiments, the pharmaceutical composition is formulated for intravenous administration.

The terms “pharmaceutically acceptable” and “pharmacologically acceptable” include molecular entities and compositions that do not produce an adverse, allergic, or other untoward reactions when administered to an animal, or human, as appropriate. For human administration, preparations should meet sterility, pyrogenicity, general safety, and purity standards as required by a government drug regulatory agency, e.g., the United States Food and Drug Administration (FDA) Office of Biologics standards.

The composition of the present invention may be administered by any known method. The term “administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, intravenously, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administration can also be performed, for example, once, a plurality of times, and/or over one or more extended periods. According to some embodiments, the composition is administered 1, 2, 3, 4, 5 or 6 times a day. According to other embodiments, the composition is administered 1, 2, 3, 4, 5 or 6 times a month. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

The term “parenteral” refers to subcutaneous, intracutaneous, intravenous, intramuscular, intraarticular, intraarterial, intrasynovial, intrasternal, intrathecal, intralesional, intraperitoneal and intracranial injection, as well as various infusion techniques.

According to another aspect, the present invention provides a method for treating allergy in a subject in need thereof comprising administering to said subject a therapeutically effective amount of engineered immune cells according to any one of the above aspects and embodiments, or a pharmaceutical composition comprising the immune cells. According to yet some embodiments, the present invention provides a method for killing B cells expressing BCR-IgE comprising administering to said subject a therapeutically effective amount of engineered immune cells according to any one of the above aspects and embodiments, or a pharmaceutical composition comprising the immune cells. All terms, embodiments and definitions disclosed in any one of the above aspects apply and are encompassed herein as well.

The term “therapeutically effective amount” of a drug or agent is an amount of a drug or an agent that, when administered to a subject will have the intended therapeutic effect, e.g., treating allergy. The full therapeutic effect does not necessarily occur by the administration of one dose and may occur only after the administration of a series of doses. Thus, a therapeutically effective amount may be administered in one or more administrations. The precise effective amount needed for a subject will depend upon, for example, the subject's size, health and age, the nature and extent of the cognitive impairment, and the therapeutics or combination of therapeutics selected for administration, and the mode of administration. The skilled person can readily determine the effective amount for a given situation by routine experimentations.

According to any one of the above embodiments, the pharmaceutical composition of the present invention is for use in treating allergy, allergic disease, condition or disorder in a subject in need thereof.

The term “treating” a condition or patient refers to taking steps to obtain beneficial or desired results, including clinical results. Beneficial or desired clinical results include, but are not limited to, ameliorating, abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting the development of symptoms characteristic of the disorder(s) being treated; (c) limiting worsening of symptoms characteristic of the disorder(s) being treated; (d) limiting recurrence of the disorder(s) in patients that have previously had the disorder(s); and/or (e) limiting recurrence of symptoms in patients that were previously asymptomatic for the disorder(s). According to some embodiments, the use comprises treating, preventing or inhibiting allergy or inhibiting or reducing IgE induced reaction. As used herein, the term “preventing” when used in relation to a condition, refers to the administration of a composition that reduces the frequency of, or delays the onset of, symptoms of a food allergy in a subject relative to a subject which does not receive the composition.

As used herein, the expression “a subject in need thereof” means a human or non-human animal that exhibits one or more symptoms or indicia of allergy or atopy, and/or who has been diagnosed with allergy to an allergen. In certain embodiments, the term “subject in need thereof” includes subjects that are at an increased risk for developing an allergy or an allergic response to an allergen. In certain embodiments, the term includes subjects that show allergen sensitization to one or more allergens. In certain embodiments, the methods of the present invention may be used to treat subjects that show elevated levels, compared with a pre-determined baseline, of one or more serum biomarkers including, but not limited to, total IgE, allergen-specific IgE, thymus and activation-regulated chemokine (TARC), pulmonary and activation-regulated chemokine (PARC), lactate dehydrogenase (LDH), and periostin. For example, the methods of the present invention comprise administering the engineered immune cells of the present invention to patients with elevated levels of allergen-specific IgE. The terms “subject” and “patient” have been used interchangeably herein.

The term “allergy” as used herein refers to IgE-associated/mediated immune response and refers also to allergic diseases and disorders. As used herein, the terms “allergic response”, “allergic reaction”, “allergic symptom”, and the like, include one or more signs or symptoms selected from the group consisting of urticaria (e.g., hives), angioedema, rhinitis, asthma, vomiting, sneezing, runny nose, sinus inflammation, watery eyes, wheezing, bronchospasm, reduced peak expiratory flow (PEF), gastrointestinal distress, flushing, swollen lips, swollen tongue, reduced blood pressure, anaphylaxis, and organ dysfunction/failure. An “allergic response,” “allergic reaction,” “allergic symptom,” etc., also includes immunological responses and reactions such as, e.g., increased IgE production and/or increased allergen-specific immunoglobulin production. The term “allergy” or “allergic disease” refers to diseases that occur due to immune reaction of the type I hypersensitivity reaction. In this reaction, a specific allergen binds its related allergen-specific IgEs, that are engaged on their high-affinity receptor FcϵRI on mast cells and basophils (the effector cells), and by cross-linking several receptors the engagement of the allergen leads to activation of the effector cells and degranulation of pre-formed mediators (such as Histamine, PAF, Heparin, proteases, chemokines, prostaglandins, leukotriens) that cause the allergic symptoms. The symptoms can be local, involving a single organ, or multisystemic, and may include swelling and itching of skin and mucosal surfaces, sneezing, bronchoconstriction and dyspnea, laryngeal edema, vomiting and diarrhea, hemodynamic changes including blood vessel dilatation, drop in blood pressure and shock. A multisystemic allergic reaction is termed “anaphylaxis” and may lead to death of the allergic person. A chronic exposure to an allergen to which the allergic person is allergic to, may lead to chronic allergic inflammation in the exposed system (skin, eyes, airways, gastrointestinal tract), inflammation that typically involves recruitment of eosinophil leukocytes.

According to some embodiments, the allergy is selected from allergic rhinitis, allergic asthma, immediate hypersensitivity drug allergy, IgE-mediated food allergies and life-threatening venom allergies.

The term “allergen”, as used herein, includes any substance, chemical, particle or composition which is capable of stimulating an allergic response in a susceptible individual. Allergens may be contained within or derived from a food item such as, e.g., dairy products (e.g., cow's milk), egg, celery, sesame, wheat, soy, fish, shellfish, sugars (e.g., sugars present on meat such as alpha-galactose), peanuts, other legumes (e.g., beans, peas, soybeans, etc.), and tree nuts. Alternatively, an allergen may be contained within or derived from a non-food item such as, e.g., dust (e.g., containing dust mite), pollen, insect venom (e.g., venom of bees, wasps, mosquitos, fire ants, etc.), mold, animal fur, animal dander, wool, latex, metals (e.g., nickel), household cleaners, detergents, medication, cosmetics (e.g., perfumes, etc.), drugs (e.g., penicillin, sulfonamides, salicylate, etc.), therapeutic monoclonal antibodies (e.g., cetuximab), ragweed, grass and birch. Exemplary pollen allergens include, e.g., tree pollens such as birch pollen, cedar pollen, oak pollen, alder pollen, hornbeam pollen, aesculus pollen, willow pollen, poplar pollen, plantanus pollen, tilia pollen, olea pollen, Ashe juniper pollen, and Alstonia scholaris pollen. Other examples of allergens can be found elsewhere herein. The terms “allergen” and “antigen” are used interchangeably through the disclosure.

According to any one of the above embodiments, the engineered immune cells of the present invention may be co-administered with any other anti-allergy or anti-inflammatory agent. The term “co-administration” refers to the administration of two or more compounds in a regimen selected from a single combined composition, separate individual compositions administered substantially at the same time, and separate individual compositions administered under separate schedules and include treatment regimens in which the agents are not necessarily administered by the same route of administration or at the same time. The term “co-administration” encompasses the administration of a first and second agent in an essentially simultaneous manner, such as in a single dosage form, e.g., a capsule or tablet having a fixed ratio of first and second amounts, or in multiple dosage forms for each. The agents can be administered sequentially in either order. When coadministration involves the separate administration of each agent, the agents are administered sufficiently close in time to have the desired effect (e.g., complex formation). The term “sequential manner” refers to an administration of two compounds at different times, and optionally in different modes of administration. The agents can be administered sequentially in either order. The term “substantially simultaneous manner” refers to the administration of two compounds with only a short time interval between them. In some embodiments, the time interval is in the range of from 0.5 to 60 minutes.

It is understood that the treatment of prevention of allergic condition and/or reaction may require long term treatment, sometimes long before the exposure to the allergen. According to some embodiments, the use of the present invention prevents allergic reaction or diminishes it. In some embodiments, the treatment may require sequential administering of the pharmaceutical composition or administering with prolonged pauses. The regiment may be assessed by CAR T cells count.

According to some embodiments, the pharmaceutical composition is administered by an injection or infusion. According to some embodiments, the treatment may be performed as a single treatment or as a multiple-dose administration.

In some embodiments, the pharmaceutical composition comprises from about 107-109 or 108-1010 engineered immune cells.

According to some embodiments, the method comprises an adoptive cell transfer (ACT) therapy. In another embodiment, the immune cells are formulated as an ACT composition. In another embodiment said immune cells are administered to said subject in the form of an ACT. As used herein, and unless otherwise specified, the terms “adoptive transfer” and “adoptive cell transfer” refer to a form of passive immunotherapy where previously sensitized immunologic agents (e.g., cells or serum) are transferred to the recipients. The phrases “adoptive transfer immunotherapy”, “adoptive cell therapy” and “adoptive cell immunotherapy” are used interchangeably herein to denote a therapeutic or prophylactic regimen or modality, in which effector immunocompetent cells, such as the engineered cell compositions of the invention, are administered (adoptively transferred) to a subject in need thereof, to alleviate or ameliorate the development or symptoms of a condition. The cells may be autologous or allogeneic. According to certain preferable embodiments, the cell composition is histocompatible with the subject to be treated. In some embodiments, the cells are T cells (e.g. obtained from peripheral blood or tumor infiltrating lymphocytes), that may be further manipulated or engineered as disclosed herein.

In another aspect there is provided a process for preparing a cell composition adapted for ACT, the process comprising:
a) providing a sample of immune cells (e.g. blood-derived immune cells),
b) activating the cells (e.g. with T-cell activating agents including, but not limited to, TCR/CD3 activators, co stimulating agents and/or cytokines)
c) engineering the cells to express a CAR as disclosed herein, and
d) expanding the cells (e.g. in the presence of T-cell activating cytokines such as IL-2) so as to obtain an effective amount of cells expressing the CAR.

In one embodiment, the blood-derived immune cells are peripheral blood mononuclear cells (PBMC). In another embodiment, step b) is performed in the presence of anti CD3 and anti CD28 antibodies for 24-72 hours and optionally IL2. In another embodiment step c) is performed using transfection and/or transduction methods known in the art, e.g. transfected by electroporation, using Nucleofector technology, liposome-mediated transfer, or transduced using a viral vector as disclosed herein. In another embodiment step d) may be performed by expanding the cells in the presence of IL-2, e.g. for at least 3, 5, or 8 days and up to e.g. 10, 12 or 15 days.

In another embodiment, step c) may be at least partly overlapping with step d). In another aspect there is provided a process for preparing a cell composition adapted for

ACT, the process comprising:
a) providing a PBMC sample,
b) activating the cells in the presence of anti CD3 and anti CD28 antibodies for 24-72 hours and optionally IL2,
c) engineering the cells to express a CAR as disclosed herein, and
d) expanding the cells in the presence of IL-2 (e.g. 100-350 U/ML) so as to obtain an effective amount of cells expressing the CAR.

According to yet another aspect, the present invention provides a chimeric antigen receptor (CAR) comprising an antigen binding domain (ABD) that binds specifically to an M1′-domain of a human IgE-BCR. All terms, embodiments and definitions disclosed in any one of the above aspects apply and are encompassed herein as well. According to some embodiments, the ABD comprises a set of 6 complementarity-determining regions (CDR) sequences, wherein the heavy chain CDR1 comprises the amino acid sequence SEQ ID NO: 6, the heavy chain CDR2 comprises the amino acid sequence SEQ ID NO: 7, the heavy chain CDR3 comprises the amino acid sequence SEQ ID NO: 8, the light chain CDR1 comprises the amino acid sequence SEQ ID NO:9, the light chain CDR2 comprises the amino acid sequence SEQ ID NO: 10, and the light chain CDR3 comprises the amino acid sequence SEQ ID NO: 11. According to some embodiments, the ABD comprises a heavy-chain variable domain (VH) comprising 3 CDRs of a VH domain having the amino acid sequence as set forth in SEQ ID NO: 13 and a light-chain variable domain (VL) comprising 3 CDRs of a VL domain having the amino acid sequence as set forth in SEQ ID NO: 12.

According to some embodiments, the ABD comprises a VH domain having the amino acid sequence as set forth in SEQ ID NO: 13 and a VL having the amino acid sequence as set forth in SEQ ID NO: 12. According to some embodiments, the ABD is a scFv comprising the amino acid sequence SEQ ID NO:14.

According to some embodiments, the ABD comprises a heavy-chain variable domain (VH) having an amino acid sequence having at least 85% sequence identity to SEQ ID NO: 13 and a light-chain variable domain (VL) having an amino acid sequence having at least 85% sequence identity to SEQ ID NO: 12, wherein the ABD comprises 6 CDRs comprising the amino acid sequences SEQ ID NO: 6-11 and wherein there is no mutation in the CDRs sequences. According to some embodiments, the ABD is an scFv comprising an amino acid sequence having at least 85% sequence identity to SEQ ID NO: 14, wherein the ABD comprises 6 CDRs comprising the amino acid sequences SEQ ID NO: 6-11 and wherein there is no mutation in the CDRs sequences.

According to some embodiments, the ABD comprises a heavy-chain variable domain (VH) having the amino acid sequence as set forth in SEQ ID NO: 13 and a light-chain variable domain (VL) having the amino acid sequence as set forth in SEQ ID NO: 12. According to any one of the above embodiments, the CAR comprises a transmembrane domain (TM domain), a costimulatory domain and an activation domain.

According to some embodiments, the TM domain is a TM domain of a receptor selected from CD28 and CD8. According to one embodiment, the TM domain is a TM domain of CD28.

According to any one of the above embodiments, the costimulatory domain is selected from a costimulatory domain of a protein selected from CD28, 4-1BB, OX40, iCOS, CD27, CD80, CD70 and a combination thereof. According to some embodiments, the costimulatory domain is a costimulatory domain of CD28. According to some embodiments, the costimulatory domain is a costimulatory domain of 4-1BB. According to some embodiments, the costimulatory domain is a costimulatory domain of OX40. According to some embodiments, the CAR comprises a costimulatory domain of CD28 and of 4-1BB. According to some embodiments, the CAR comprises a costimulatory domain of CD28 and of OX40.

According to any one of the above embodiments, the TM domain and the costimulatory domain are both derived from CD28. According to any one of the above embodiments, the antigen binding domain is linked to the TM domain via a spacer. According to any one of the above embodiments, the activation domain is selected from FcRγ and CD3-ζ activation domains. According to some embodiments, the activation domain is CD3-ζ. According to some embodiments, the activation domain is from FcRγ. According to some embodiments, the present invention provides a chimeric antigen receptor (CAR) comprising a transmembrane domain (TM domain), a costimulatory domain, an activation domain and a leading peptide, wherein the TM domain comprises the amino acid sequence SEQ ID NO: 16, the costimulatory domain comprises the amino acid sequence SEQ ID NO: 17, the activation domain comprises the amino acid sequence SEQ ID NO: 18 and the leading peptide comprises the amino acid sequence SEQ ID NO: 19. According to some embodiments, the CAR comprises a TM domain, a costimulatory domain comprising, and an activation domain, wherein the TM domain comprises an amino acid sequence having at least 85% sequence identity to SEQ ID NO: 16, and/or a costimulatory domain comprises an amino acid sequence having at least 85% sequence identity to SEQ ID NO: 17, and/or an activation domain comprises an amino acid sequence having at least 85% sequence identity to SEQ ID NO: 18 and/or a leading peptide comprises an amino acid sequence having at least 85% sequence identity to SEQ ID NO: 19. According to some embodiments, the sequence identity is at least 90% at least 92%, at least 95% at least 98% or at least 99%.

According to any one of the above embodiments, the CAR comprises the amino acid sequence SEQ ID NO: 20. According to some embodiments, the CAR comprises an amino acid sequence having at least 85% sequence identity SEQ ID NO: 20, wherein the CAR comprises an ABD comprising 6 CDRs comprising the amino acid sequences SEQ ID NO: 6-11 and wherein there is no change in the CDRs sequences. According to some embodiments, the sequence identity is at least 90% at least 92%, at least 95% at least 98% or at least 99%.

According to yet another aspect, the present intention provides a nucleic acid molecule encoding the CAR according to any one of the above aspects and embodiments. All terms, embodiments and definitions disclosed in any one of the above aspects apply and are encompassed herein as well.

The term “nucleic acid” refers to single stranded or double stranded sequence (polymer) of deoxyribonucleotides or ribonucleotides. In addition, the polynucleotide includes analogues of natural polynucleotides, unless specifically mentioned. According to an embodiment, the nucleic acid may be selected from the group consisting of deoxyribonucleic acid (DNA), ribonucleic acid (RNA), peptide nucleic acid (PNA), locked nucleic acid (LNA), and analogues thereof, but is not limited thereto. The term encompasses DNA, RNA, single stranded or double stranded and chemical modifications thereof.

The term “nucleic acid” refers to single stranded or double stranded sequence (polymer) of deoxyribonucleotides or ribonucleotides. In addition, the polynucleotide includes analogues of natural polynucleotides, unless specifically mentioned. According to an embodiment, the nucleic acid may be” selected from the group consisting of deoxyribonucleic acid (DNA), ribonucleic acid (RNA), peptide nucleic acid (PNA), locked nucleic acid (LNA), and analogues thereof, but is not limited thereto. The term encompasses DNA, RNA, single stranded or double stranded and chemical modifications thereof.

The term nucleic acid refers also the to a homolog of specific sequences. The terms “homolog” “variant”, “DNA variant”, “sequence variant” and “polynucleotide variant” are used herein interchangeably and refer to a DNA polynucleotide having at least 85% sequence identity to the parent polynucleotide. The variant may include mutations such as deletion, addition or substitution such that the mutations do not change the open reading frame and the polynucleotide encodes a peptide or a protein having substantially similar structure and function as a peptide or a protein encoded by the parent polynucleotide. According to some embodiments, the variants are conservative variants. The term “conservative variants” as used herein refers to variants in which a change of one or more nucleotides in a given codon position results in no alteration in the amino acid encoded at that position. Thus, the peptide or the protein encoded by the conservative variants has 100% sequence identity to the peptide or the protein encoded by the parent polynucleotide. According to some embodiments, the variant is a non-conservative variant encoding to a peptide or a protein being a conservative analog of the peptide of the protein encoded by the parent polynucleotide. According to some embodiments, the variant has at least at least 90%, at least 95%, at least 98% or at least 99% sequence identity to the parent polynucleotide.

It should be clear that in the case that the CAR comprises more than one polypeptide, each polypeptide can be encoded by one or more polynucleotides and the term “a nucleic acid” may refer to more than one nucleic acid encoding said polypeptides.

According to some embodiments, the nucleic acid encodes a CAR comprising the amino acid sequences SEQ ID NO: 12 and 13. According to some embodiments, the nucleic acid encodes a CAR comprising the amino acid sequence SEQ ID NO: 14. According to some embodiments, the nucleic acid encodes a CAR comprising a VH domain having an amino acid sequence having at least 85% sequence identity to SEQ ID NO: 13 and a VL domain having an amino acid sequence having at least 85% sequence identity to SEQ ID NO: 12, wherein the ABD comprises 6 CDRs comprising the amino acid sequences SEQ ID NO: 6-11 and wherein there is no mutation in the CDRs sequences. According to some embodiments, the nucleic acid encodes a CAR comprising ABD being an scFv comprising an amino acid sequence having at least 85% sequence identity to SEQ ID NO: 14, wherein the ABD comprises 6 CDRs comprising the amino acid sequences SEQ ID NO: 6-11 and wherein there is no change in the CDRs sequences. According to some embodiments, the sequence identity is at least 90% at least 92%, at least 95% at least 98% or at least 99%.

According to some embodiments, the nucleic acid encodes a CAR comprising the amino acid sequence SEQ ID NO: 20. According to some embodiments, the nucleic acid encodes a CAR comprising an amino acid sequence having at least 85% sequence identity SEQ ID NO: 20, wherein the CAR comprises an ABD comprising 6 CDRs comprising the amino acid sequences SEQ ID NO: 6-11 and wherein there is no mutation in the CDRs sequences. According to some embodiments, the sequence identity is at least 90% at least 92%, at least 95% at least 98% or at least 99%.

According to some embodiments, the nucleic acid molecule comprises the nucleic acid sequences SEQ ID NO: 21 and 22. According to some embodiments, the nucleic acid molecule comprises a nucleic acid sequences having at least 85% sequence identity to SEQ ID NO: 21 and/or 22. According to some embodiments, the nucleic acid molecule comprises the nucleic acid sequence SEQ ID NO: 23. According to some embodiments, the nucleic acid molecule comprises a nucleic acid sequences having at least 85% sequence identity to SEQ ID NO: 23. According to some embodiments, the nucleic acid molecule comprises the nucleic acid sequence SEQ ID NO: 23 and at least one of the nucleic acid sequences SEQ ID NO: 25, 26, 27 and 28. According to some embodiments, the nucleic acid molecule comprises a nucleic acid sequences having at least 85% sequence identity to SEQ ID NO: 23 and/or at least one of the nucleic acid sequences having at least 85% sequence identity to a nucleic acid sequence selected from SEQ ID NO: 25, 26, 27 and 28. According to some embodiments, the nucleic acid molecule comprises the nucleic acid sequence SEQ ID NO: 29. According to some embodiments, the nucleic acid molecule comprises a nucleic acid sequences having at least 85% sequence identity to SEQ ID NO: 29. According to any one of the above embodiments, the encoded CAR and/or ABD comprises 6 CDRs comprising the amino acid sequences SEQ ID NOs: 6-11 and wherein there is no mutation in the CDRs sequences.

According to yet another aspect, the present invention provides a nucleic acid construct comprising the nucleic acid molecule according to any one of the above aspects and embodiments, operably linked to a promoter. All terms, embodiments and definitions disclosed in any one of the above aspects apply and are encompassed herein as well.

The terms “operably linked”, “operatively linked”, “operably encodes”, and “operably associated” are used herein interchangeably and refer to the functional linkage between a promoter and nucleic acid sequence, wherein the promoter initiates transcription of RNA corresponding to the DNA sequence. A heterologous DNA sequence is “operatively associated” with the promoter in a cell when RNA polymerase which binds the promoter sequence transcribes the coding sequence into mRNA which then in turn is translated into the protein encoded by the coding sequence.

According to yet another aspect, the present invention provides a vector comprising the nucleic acid molecule or the construct according to any one of the above aspects and embodiments. All terms, embodiments and definitions disclosed in any one of the above aspects apply and are encompassed herein as well.

The terms “vector” and “expression vector” are used herein interchangeably and refer to any viral or non-viral vector such as plasmid, virus, retrovirus, bacteriophage, cosmid, artificial chromosome (bacterial or yeast), phage, binary vector in double or single stranded linear or circular form, or nucleic acid, sequence which is able to transform host cells and optionally capable of replicating in a host cell. The vector may be integrated into the cellular genome or may exist extrachromosomally (e.g., autonomous replicating plasmid with an origin of replication). The vector may contain an optional marker suitable for use in the identification of transformed cells, e.g., tetracycline resistance or ampicillin resistance. A cloning vector may or may not possess the features necessary for it to operate as an expression vector.

According to yet another aspect, the present invention provides a cell comprising the nucleic acid molecule, construct or vector of the present invention. According to some embodiments, the cell is a prokaryotic cell. According to some embodiments, the cell is a eukaryotic cell. According to some embodiments, the cell expresses or capable of expressing the CAR as defined in any one of the above aspects and embodiments. According to some embodiments, the cell is an immune cell. According to some embodiments the immune is selected from T cells, natural killer cells and macrophage.

According to yet another aspect, the present invention provides a use of a plurality of immune cell engineered to express a chimeric antigen receptor (CAR) that binds specifically to an epitope within an amino acid sequence of an extracellular domain of a human IgE-BCR that is absent from a freely circulating IgE, for the preparation of a medicament for treating allergy, allergic disease condition or disorder. All terms, embodiments and definitions disclosed in any one of the above aspects apply and are encompassed herein as well. According to some embodiments, the ABD specifically binds to the M1′-domain of a human BCR-immunoglobulin E (IgE). According to some embodiments, the CAR comprising an ABD comprising a VH having the amino acid sequence as set forth in SEQ ID NO: 13 and a VL having the amino acid sequence as set forth in SEQ ID NO: 12. According to some embodiments, the ABD is an scFv comprising the amino acid sequence SEQ ID NO: 14. According to some embodiments, the CAR comprises the amino acid sequence SEQ ID NO: 20. According to some embodiments, the immune cells are selected from T cells, natural killer (NK) cells and macrophages. According to some embodiments, the immune cells are T cells.

The following terms are relevant to the following aspects and embodiments, and are contemplated, embedded and are part of each one of the relevant embodiments in which these terms are mentioned or used.

As used herein, the forms “a”, “an” and “the” include singular as well as plural references unless the context clearly dictates otherwise. For example, the term “a protein” includes one or more copies of the recited protein.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

The following examples are presented in order to more fully illustrate some embodiments of the invention. They should, in no way be construed as limiting the scope of the invention

### EXAMPLES

### Materials and Methods

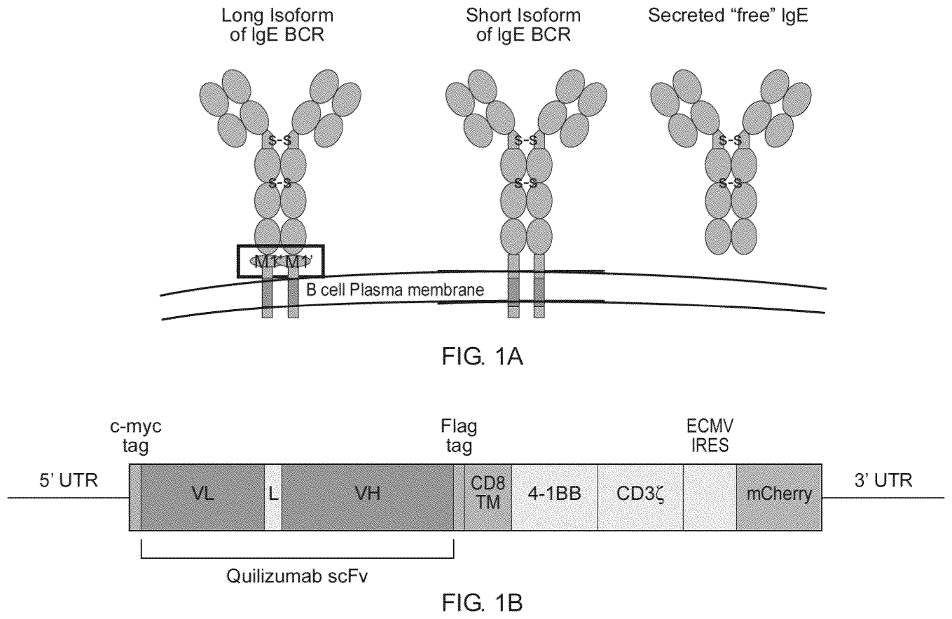

A 2nd generation CAR was designed to target the M1′ domain, a domain of IgE-BCR that does not exist in free IgE (FIG. 1A). The M′ domain is unique to the longer isoform of the two known human IgE-BCR isoforms and has the amino acid sequence SEQ ID NO: 1 (GLAGGSQSQRAPDRVLCHSGQQQGLPRAAGGSVPHPRCHCGAGRADWPGPP).

The anti-M1′ scFv sequence of the CAR was adopted from the sequence of the variable-light and variable-heavy chains of Quilizumab monoclonal antibody (disclosed e.g., in U.S. Pat. No. 8,071,097).

The CAR-scFv is connected by a hinge at its C-terminus to a CD8a transmembrane domain followed by a 4-1BB costimulatory domain, and at C terminus CD3 ζ signaling domain. The CAR is termed Q-CAR. The CAR is also denoted as anti-IgE-BCR CAR.

### CAR Vector

For transduction to human lymphocytes, the CAR was encoded on retroviral vectors MSGV1 and MSGV. In addition to the CAR, the CAR-vector encodes for a Myc-tag at the N-terminus and a Flag-tag at the C-terminus, as shown in FIG. 1B.

To ensure safety of the treatment, a coding for caspase gene fused to an inducible dimerization domain can be added to the vector. The default is the expression of an inactive monomeric caspase. However, if an adverse outcome is seen and quick shutoff is desired, the addition of a small membrane permeable molecule allows the dimerization of the caspase and the subsequent induction of apoptosis. Apoptosis induced by caspase dimerization is independent of cell cycling and is quick. The caspase is non-immunogenic and has little bystander effects compared to common alternatives for induced cell death. Similar systems were safely and successfully used in clinical trials for the elimination genetically modified T cells.

### Transfection and Transduction of Human T Lymphocytes

Retroviral vectors were first transfected to a self-packaging 293Vec-Galv cells line (Biovec pharma) assisted by in-house produced PEI transfection agent. 48 hr later, the supernatant was collected and adsorbed to a second self-packaging cell line—293Vec-RD114 (Biovec pharma). From these constitutively pseudo-virus-producing plates supernatant was collected and adsorbed to RetroNectin coated plates, to which activated human lymphocytes were applied 48 hr post activation.

For RetroNectin coating, 24-well non-coated plates (Greiner Bio-One) were used and coated with RetroNectin (Takara) following manufacturer's instructions. For inoculation of the pseudo-virus, 2 ml of filtered (0.45 micron pores) supernatant from the 293Vec-RD114 plates was applied to each RetroNectin-coated well, and centrifuged at 2000 g for two hours at 32° C. Supernatant was then aspirated and the activated lymphocytes were applied, 1×10⁶ activated lymphocytes/ml, 1 ml/well, for transduction, assisted by transduction agent Polybrene (8 microgram/ml). The plates were then centrifuged for an additional 10 minutes at 1000 g.

### Activation of Human T Lymphocytes

Single donor Peripheral Blood Mononuclear Cells (PBMCs) obtained from fresh blood were purchased from the national blood bank. PBMCs were separated using Ficoll gradient and kept stored in aliquots in liquid nitrogen.

Thawed aliquots were washed and resuspended 1×106/ml in T cell medium: MEM-alfa (Sigma), 20% heat inactivated fetal calf serum (Sigma), 1% antibiotics Pen-Strep, 50 IU/ml rhIL-2 (PeproTech),

For T cell activation, anti-human CD3 (OKT3, PeproTech) 1 microgram/ml and anti-human-CD28 (PeproTech) 1 microgram/ml was added.

Transduction efficiency by flow cytometry was assessed by detection of mCherry or staining with anti-myc antibodies for flow cytometry, and enumeration.

### Engineered Target Cells

Since the target IgE⁺-B cells (B cells expressing IgE-BCR) are extremely sparse in blood, even in the blood of an allergic patient, an initial construct that included solely the target sequence M1′ (Exons M2-to M1′) was designed, produced, and transduced via Lentiviral transduction to a variety of human and mouse cell lines. Later, a larger construct of IgE heavy chain constant domain, Exons 2-M2, i.e., with M1′ was lentivirally transduced into B cell lines (NALM6, Daudi, U266), to serve as a surrogate construct to the long isoform of the IgE-BCR. Non-transduced B cells of the same lines serve as a control, and as an additional control served the same cell lines transduced with a surrogate construct for the short isoform of human IgE-BCR, IgE heavy chain constant domain, Exons 2-M2 i.e., without the M1′.

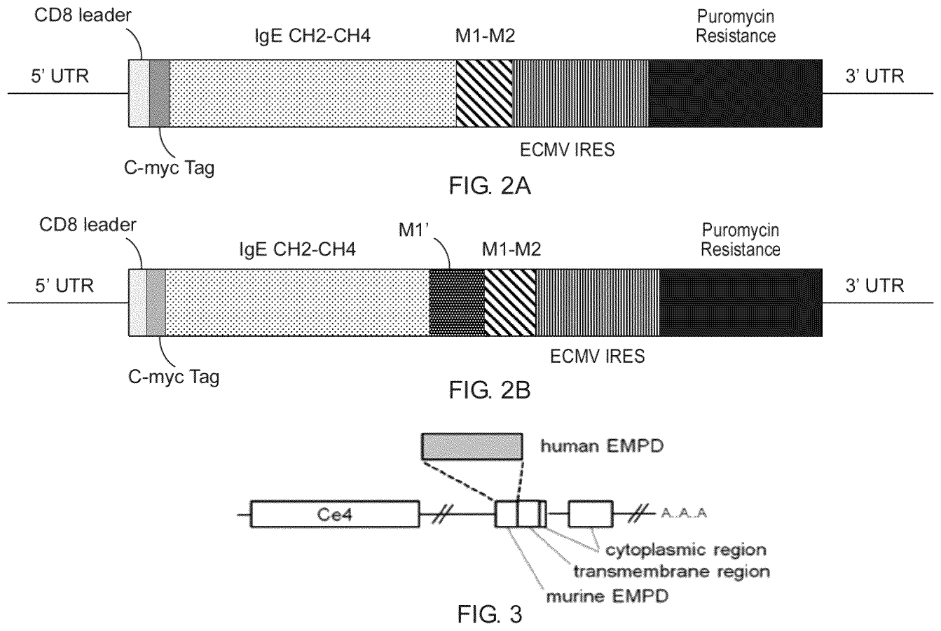

A schematic representation of the short isoform of IgE-BCR surrogate construct lacking M1′ that was transduced into a NALM6 cell line is shown in FIG. 2A. FIG. 2B shows a schematic representation of the “Long IgE-BCR surrogate construct”.

### In Vitro Induction of IgE+ Human B Lymphocyte

Human B cells were separated from human PBMCs or human tonsils and adenoids (obtained through a collaboration with the ENT department, Sheba Medical Center) using EasySep kit by Stemcell.

Since the prevalence of IgE+ B cells in blood is extremely low, to study CAR-Ts activation and killing of primary human IgE+ B cells in vitro, human B cells under different reference induction protocols with IL-4 and anti-cd40 were used, to achieve prominent IgE+ B population to serve as a target population for the CAR.

### T Cells Activation and Killing Assay

After 6-24 h of co-culture with target populations of cells, supernatant was collected and assayed with an ELISA kit for human IFN-gamma production (R&D systems) and by flow cytometry for CD69 expression. The killing of target cells was evaluated using the Luciferase killing assay (see Example 1 hereinbelow) and by assessing the diminishing proportion of target populations as detected by flow cytometry (stained with Anti-hCD19-FITC, clone HIB19, Biolegend, Anti-IgE-BV, clone G7-26, BD Horizon, Quilizumab-APC Creative Biolabs).

### Mast Cell Degranulation Assay

To exclude the possibility of CAR-T activation by FcϵRI-bound IgE on mast cells and basophils co-culture experiments were performed with LAD2 immortalized mast cells that had been pre-incubated with free hIgE. Specifically, LAD2 mast cells (originally established from bone marrow aspirates from a patient with mast cell sarcoma/leukemia) were maintained in 1 million/mL of StemPro-34 SFM (Invitrogen) supplemented with 2 mM 1-glutamine, 50 μg/ml streptomycin, 100 IU/ml penicillin, and 100 ng/ml recombinant human stem cell factor (SCF) (PeproTech, Rocky Hill, NJ). Mast cell degranulation following the co-culture was monitored by β-hexosaminidase activity using the PNAG substrate.

Specifically, on the day before experiment, free hIgE (Abcam ab65866) was added to the LAD2 culture at a final concentration of 1 μg/mL. Co-culture experiment was performed as follows: In Eppendorf tubes, LAD2 (after 3×PBS wash) were plated (duplicates) 100,000 per 200/microliters medium well, either alone or with 100,000 T cells or Q-CAR-T cells (average transduction rate 50%), and incubated in 37° C. for 30 minutes. As a positive control, LAD2 were cultured with 10 microgram/mL anti-human-IgE (Mabtech Following the co-culture, Mast cell degranulation was monitored by β-hexosaminidase activity: Eppendorf tubes underwent spin down 30 sec and 20-μl aliquots of co-culture supernatants were incubated for 90 min at 37° C. with 50 μl of substrate solution consisting of 1.3 mg/ml p-nitrophenyl-N-acetyl-β-d-glucosaminide (PNAG) (Sigma) in 0.1 M citrate buffer (pH 4.5). For total granules content of LAD2 cell assay was performed in parallel with 20-μl of cell lysates (lysis with Triton20 1:200). Reactions were stopped by the addition of 150 μl of 0.2 M glycine (pH 10.7). OD was read at 405 nm (and 620 nm read of background) using an ELISA plate reader. Results (mean±SD) were expressed as percentage of total β-hexosaminidase activity present in the cells.

### In Vivo Experiments

In vivo tumor expansion assay in immunodeficient NOD.Cg-PrkdescidIl2rgtm1Wjl/SzJl “NSG” mice.

Mice were housed in the Tel Aviv University animal facility under specific pathogen-free conditions. Groups of up to five mice per IVC cage (Lab-Products) were housed on a 12 h light/dark cycle, on autoclaved ASPEN wood chips bedding, at an ambient temperature of 22° C.±1° C., with humidity controlled at 50%, had ad libitum access to regular laboratory chow (Altromin1324; Altromin, Lage, Germany), and were provided with UV-irradiated and micro-filtered Hydropac system for water. Animal experiments were approved by the animal care and use committee (IACUC) of Tel Aviv University (approval protocol no. TAU-LS-IL-2401-104-5) and conducted in accordance with NIH guidelines.

For animal studies and bioluminescence imaging NSG male mice 6- to 8-week-old were used in all experiments. NALM6-Long-IgE-Luciferase+ cells were systemically injected IV to tail vein (1×10⁶ cells/mouse). To monitor tumor growth, mice were i.p. injected with 150 mg/kg D-luciferin (BioGold) and imaged using the IVIS system (Xenogen) 10 min after injection. Upon appearance of a visible signal (day 6 post tumor injection), Q-CAR T cells or untransduced control T cells were systemically injected IV to tail vein (2×10⁶ cells/mouse). Mice were imaged twice weekly. An untreated group served as another negative control. The bioluminescence images were analyzed using Living Image software 4.7.3 (Caliper Life Sciences). Student's t test statistical analysis and graphing were performed using Excel/GraphPad Prism 8 software for MAC. Data represent mean±SEM, n values are listed in Example 5 hereinbelow.

Engineering Transgenic Mouse to Express hIgE Constant Domain

Unlike humans, mice do not possess two IgE-BCR isoforms, and specifically the M1′ domain. A transgenic mouse was designed in which in the IgE constant domain the EMPD (extra membranal proximal domain) of the mouse is replaced by a human one (as shown in FIG. 3 based on Vigl, B, et al., J. Immunol. Methods, 2017, 449, 28-36). Mice were designed with homozygous replacement of the endogenous murine short EMPD by the human long EMPD, by use of CRISPR/Cas9 system.

Knock-out of murine EMPD were prepared using the following two sgRNA:

mH59: (SEQ ID NO: 30)
GGTCCTGGAGGTCTAGCTCT
mH57: (SEQ ID NO: 31)
GCGAGGAGCTGGAAGAGCTG
Donor ssDNA (SEQ ID NO: 32):
CTGTCACCTGGAGTCTGGGGAAGCTAACTGGCTGGTCCCACCCCATCCC
Agggctggctggcggctccgcgcagtcccagagggccccggatagggtg
ctctgccactccggacagcagcagggactgccgagagcagcaggaggct
ctgtcccccacccccgctgccactgtggagccggggggctgactggcca
ggtcccccagagctggacgtgtgcgtggaggaggccgagggcgaggcgc
cgtggCTGTGGACCAGTATTTGTGTCTTCATCACCCTGTTCCTGCTCAG
TGTGAG

The transgenic mouse was produced in a BALBc strain background—a strain that is commonly used in allergy models due to higher propensity of these mice to develop allergy.

Experiments in Immunocompetent Transgenic Mice with BALBc Background:

High IgE production by the mice is induced in two ways: by injection of goat anti-mouse IgD antibodies, that are known to provoke a polyclonal reaction and hyper production of all isotypes, and by sensitization to a specific allergen (OVA, i.e., hen's egg allergy). The ability of mouse Q-CAR T treatment to eliminate IgE production and prevent anaphylaxis while providing the adoptive transfer of engineered cells is assessed either before (prophylactically) or after sensitization (therapeutically).

Control groups of mice are adoptively transferred with T cells engineered to express an irrelevant CAR. First, the numbers of CAR-T cells in blood, in treated and control mice are monitored using flow cytometry. The levels of free IgE are followed using ELISA. In addition, the following parameters are monitored: 1) the percentage of IgE-binding peritoneal mast cells as well as that of IgE-binding basophils in the bone marrow and spleen; 2) percentage and immune-phenotype of splenic B cells, using quilizumab mAb to detect cells expressing the long isoform of IgE; 3) the IgE response to OVA sensitization; 4) the IgE response to goat anti-IgD immunization 5) prevention of anaphylaxis triggered by active sensitization and chalange with Ova proteins. The clinical reactions, side effects and off target effects are followed as well. The weight, appearance and behavior are scored and ELISA is used to monitor blood levels of IL6, IL8, IL10, TNFα, IFNγ, AST, ALT, CRP and ferritin. Quilizumab antibodies for flow cytometry are used to detect and evaluate the presence of the long IgE-BCR isoform while common anti-IgE antibodies are used to detect total-IgE-BCR.

Food Allergy Model Sensitization and Challenge with OVA Allergen

### Sensitization:

Mice are intraperitoneally injected with 100 μl of OVA solution (1 mg/ml of OVA with 1 μg/ml of Pertussis toxin and 10 mg/ml of aluminum potassium sulfate as adjuvants). The control solution is identical to the test item but without OVA. The sensitization is performed 18-21 days before challenge.

### Challenge

Mice are injected with 50 μl of OVA solution (500 μg of OVA) intraperitoneally or intravenously. Anaphylaxis is monitored by temperature measurements, clinical scoring, and evaluating levels of Mast cell protease-1 (MCPT-1) levels in serum of mice collected 1 hour after challenge, by ELISA. In sacrificed animals, the following parameters are measured: the rate and immune phenotype of M1′+IgE expressing B cells in the spleen and the total IgE on FcεRI+, c-Kit+ mast cells in the spleen and in the jejunal lamina. Ultimately, survival is monitored in treated and mock-treated controls.

### Example 1

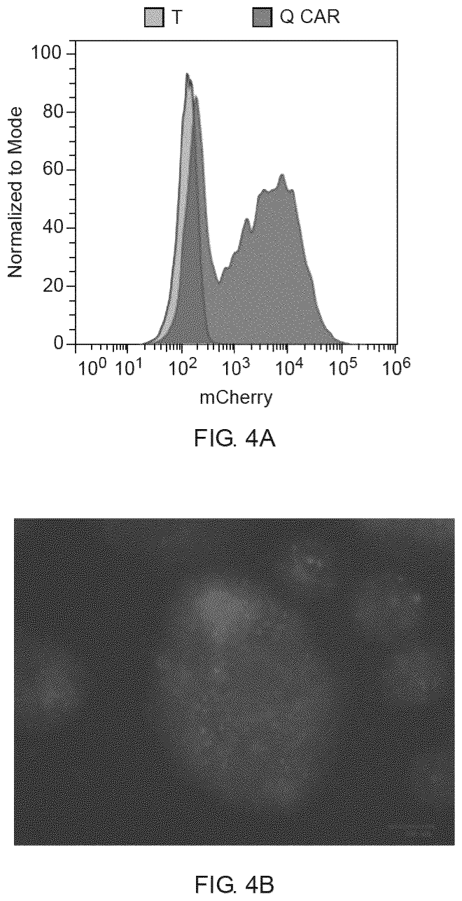

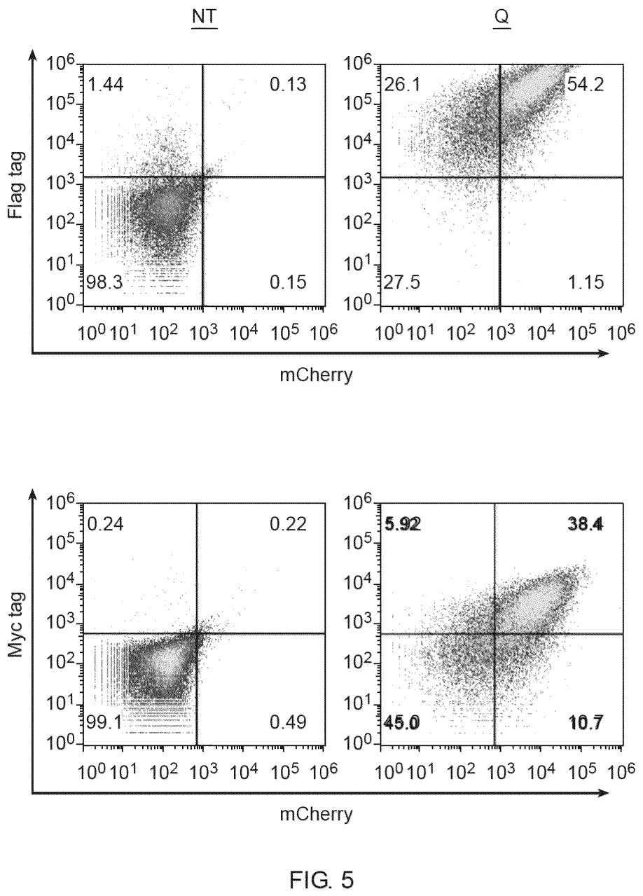

CARs were expressed on human primary T lymphocytes (as well as on Jurkat T cell line), via retroviral transduction. A good transduction of CAR into lymphocytes was achieved with an estimated average percent of CAR expression on lymphocytes of 60% and above. FIG. 4A shows a representative histogram overlay from flow cytometry data with and without transduction of Q CAR. FIG. 4B shows fluorescence microscope imaging showing mCherry expression in transduced human lymphocytes. FIG. 5 shows that detection of mCherry by flow cytometry correlates with staining anti-FLAG staining (top) and anti-Myc staining (bottom), further confirming the Q CAR construct. On left, untransduced human lymphocytes; on right, human lymphocytes transduced with Q CAR.

In preliminary experiments, only M1′ fragment was expressed on target cell lines (Daudi and 293T HEK). Hereinbelow, it is demonstrated that a co-culture with primary T cells or Jurkat cell line transduced with the anti-BCR-IgE CAR (SEQ ID NO: 20) leads to activation of the cells expressing the CAR. Specifically, an increase in IFN-gamma production and up-regulation of CD69 expression was shown.

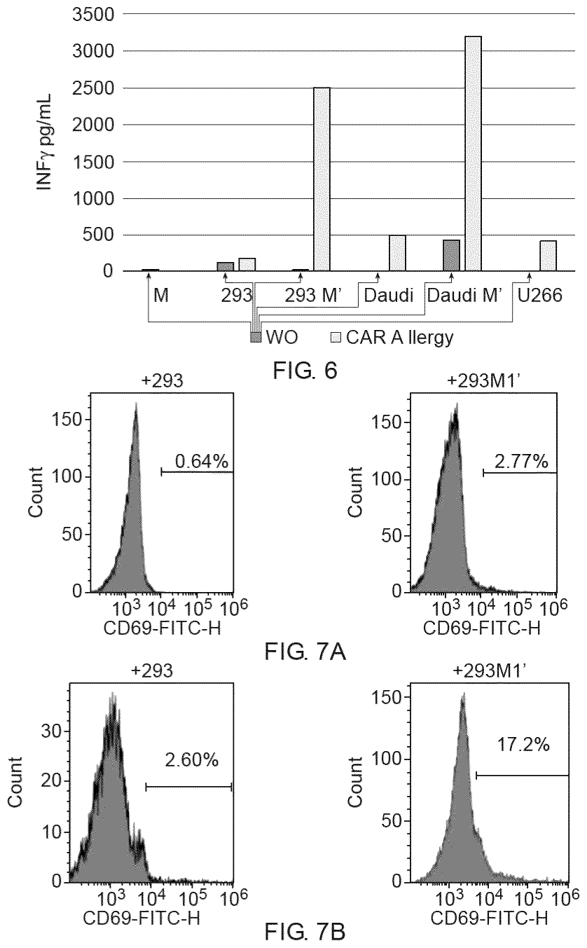

FIG. 6 shows the activation of primary human CAR-T cells by human cell lines expressing the M1′ segment as evidenced by a significant increase in IFN-gamma production.

Primary human T cells and Q-CAR-T cells of the same donor, were co-cultured with cells of two cell lines HEK293 and Daudi cell lines (served as control), and with the same cell lines engineered to express M1′. Activation of the Q-CAR-T cells was evident by increased IFN-gamma production upon co-culture with cell lines expressing M1′. No activation was observed when the Q-CAR-T cells were incubated with control cell-lines not expressing M1′ or with IgE-secreting myeloma cell line U266 that does not express IgE-BCR. The regular T cells, designated as WO (=without the CAR) were not activated upon co-culture with any of these cell lines.

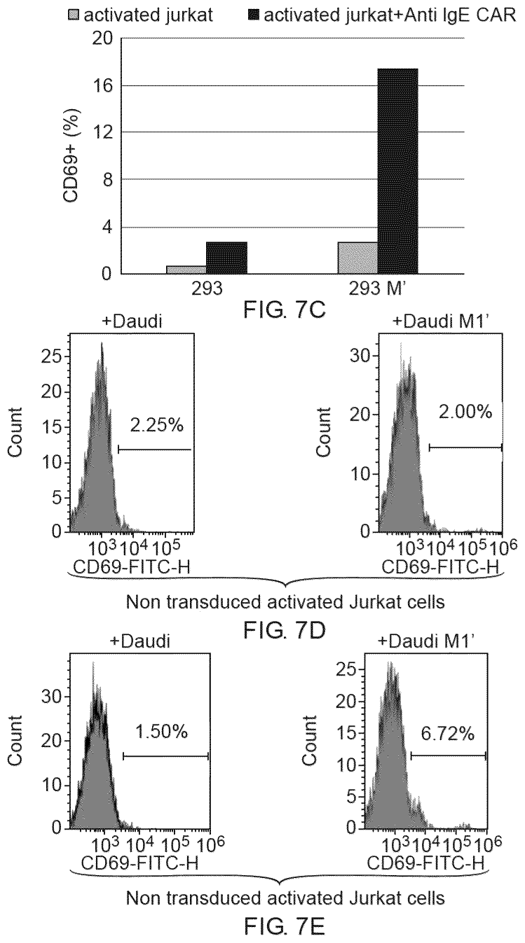

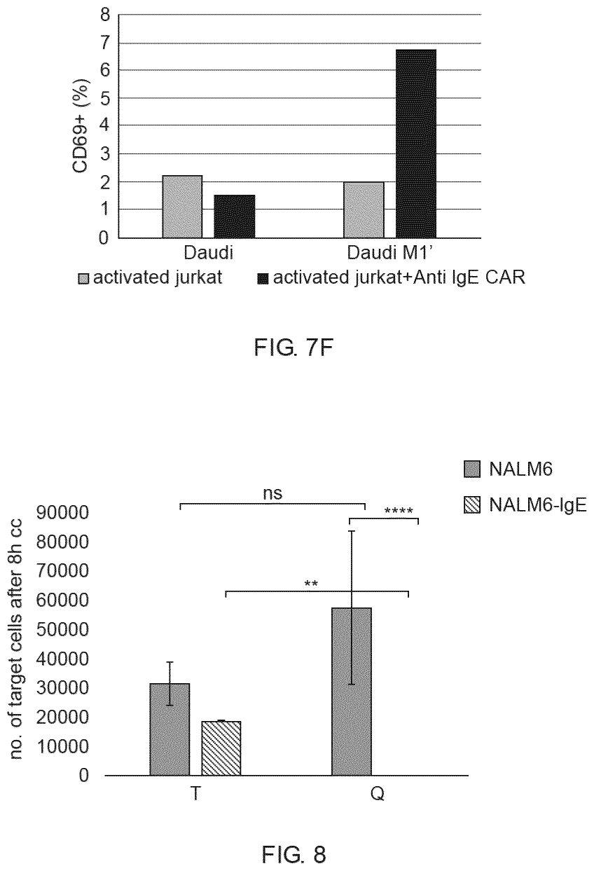

Flow cytometry analysis of the CD69 activation marker expressed by regular Jurkat cells (FIG. 7A) and by Jurkat cells transduced with Q CAR (FIG. 7B) upon co-culture with either regular HEK293 cells (left) or the same cell line expressing the M1′ segment (right), data represented also in a bar-graph (FIG. 7C). Flow cytometry analysis of the CD69 activation marker expressed by regular Jurkat cells (FIG. 7D) and by Jurkat cells transduced with anti-IgE-BCR CAR (FIG. 7E) upon co-culture with either regular Daudi cell line (left) or the same cell line expressing the M1′ segment (right), data represented also in a bar-graph (FIG. 7F).

Encouraged by the preliminary results, NALM6 and U266 human B cell lines were engineered to express “IgE-BCR surrogate construct” (FIG. 2A and FIG. 2B). FIG. 2A is the short construct lacking M1′ and FIG. 2B is the long construct including M1′.

The potency of the therapy against several engineered cell lines was tested and due to non-specific alloreactivity and technical issues, experiments were continued with the NALM6 cells.

Co-culture of CAR-T cells was performed with target cells and activation assessed by IFN-gamma production. Killing ability was assessed via flowcytometry analysis of target cell populations before and after co-culture with effector cells, and via a Luciferase assay. The killing assay via the Luciferase activity was performed with target cell lines transduced with lentivirus to express the Luciferase firefly enzyme. The co-culture experiments were performed under different conditions-varying time span (7-24 hours) or varying Effector:Target (E:T) ratios.

### Killing:

### Killing Assessed by Luciferase Killing Assay

In a 96-well plate, in technical duplicates, 200K/well Q-CAR T cells were co-cultured for 13 h E:T 2:1 with NALM6(Luc+) or NALM6-L(Luc+) in 200 microliter RPMI medium supplemented by 10% FBS 1% PS antibiotics. Control experiments were held in parallel with non-transduced T (activated, same donor). n=3. At the end of co culture, the plate was centrifuged at 500 g for 5 min, and 100 microliters of supernatant was removed and replaced by Luciferin containing buffer (“ONE-Glo”, Promega). The plate was read after 3 minutes and luminescence measured. As a standard scale plates with an escalating measured number of cells were plated. As shown in FIG. 8, within 13 hours Q-CAR T cells completely eliminated the NALM6-L cells (here termed NALM6-IgE), while elimination of NALM6 was similar to elimination by non-transduced T cells.

### Killing Assessed by Flowcytometry Analysis

In another experiment, killing of target populations by effector T and Q-CAR-T cells was assessed by flow cytometry analysis using the following gating strategy:

Staining was performed at starting point and at the end point of the co-culture.

Different cell populations were designated as:
“Targets”—cells stained with anti hCD19 (NALM6/NALM6-S/NALM6-L).
“Effectors”—the non-CD19+ cells in the co-culture i.e., T cells and CAR-T cells (in parallel these cells were stained with anti hCD3)
CAR-T cells-express mCherry
IgE-BCR-surrogate construct+ cells—stained with anti-h-IgE

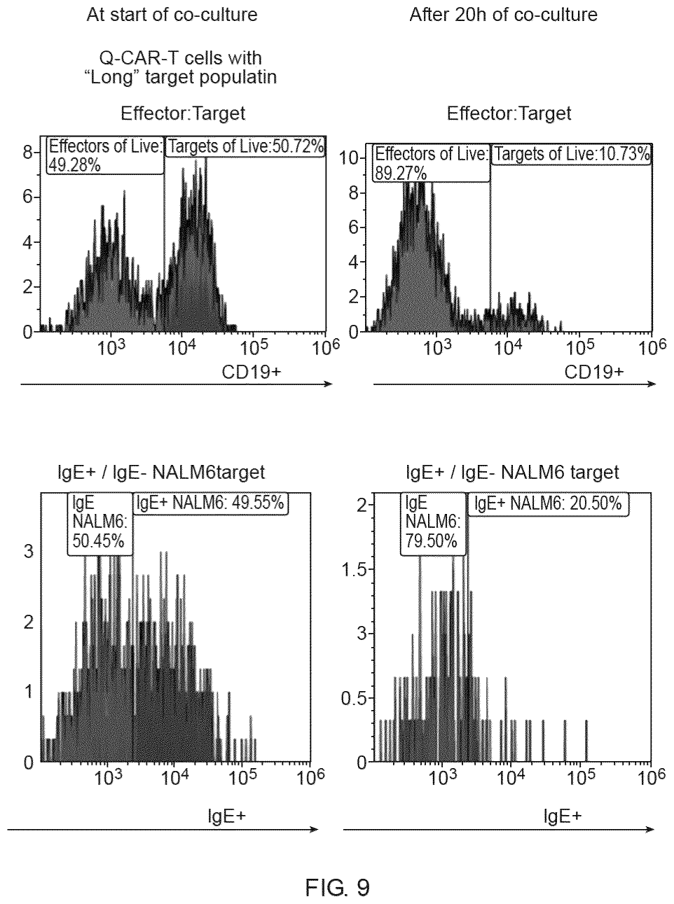

Presented is an example of flowcytometric data of a co-culture experiment that demonstrates the efficient and specific killing ability of the Q-CAR-T cells, also referred to as “Q” (for Quilizumab-based); with FIG. 9 showing co-culture of 20 hours duration, E:T ratio of 1:1, CAR transduction 83%. Specifically, FIG. 9 shows results of a flow cytometry analysis of a co-culture experiment Q-CAR-T cells (“Q”) with a mixed population of NALM6/Long mIgE-NALM6-L, showing the prominent elimination of the IgE+ population among the mixed target population after 20 hours of co-culture.

### Activation:

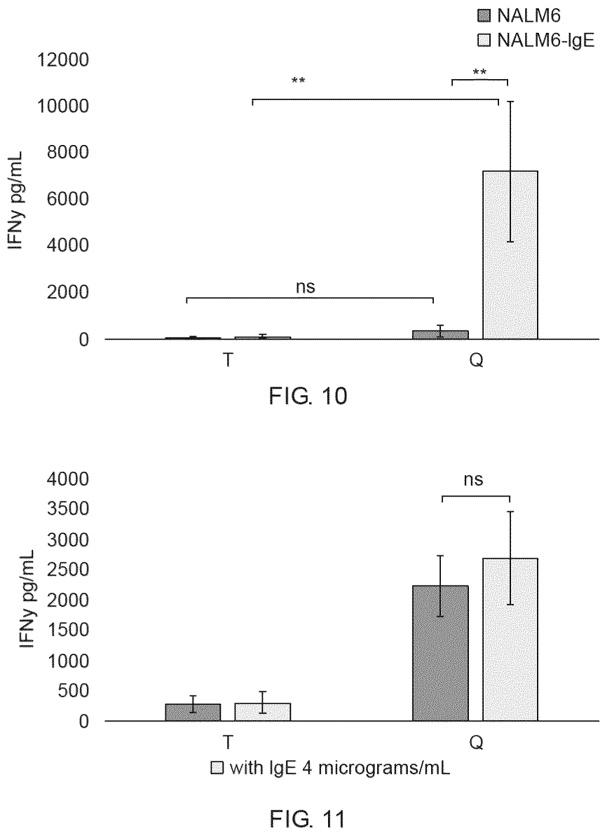

In a 96-well plate, in technical duplicates, 100K/well Q-CAR T cells were co-cultured for 8 h E:T 1:2 with NALM6(Luc+) or NALM6-L(Luc+) in 200 microliter RPMI medium supplemented by 10% FBS 1% PS antibiotics. Control experiments were held in parallel with non-transduced T (activated, same donor). n=3. At the end of co-culture, the plate was centrifuged at 500 g for 5 min, 100 microliters of the supernatant were aspirated and IFNgamma was measured by ELISA (kit Human IFN-gamma DouSet ELISA, R&D systems). FIG. 10 shows the specific activation of Q-CAR T cells only upon co-culture with NALM6-L expressing IgE with M1′, and not with NALM6, while T cells were not activated at all.

### In Conclusion:

Q-CAR-T cells designed to target M1′ successfully eliminated NALM6 cells expressing long-IgE-BCR surrogate construct (NALM6-L or NALM6-IgE) but not wild type NALM6 or NALM6 that express the short isoform of IgE-BCR-surrogate construct (NALM6-S). This targeted elimination was accompanied by an increase of IFN-gamma production by the effector cells.

### Example 2

These experiments assessed whether high levels of free-IgE in the culture media, as is present in the blood of atopic patients, affects the potency of the CAR T cells of the present invention comprising a CAR having the amino acid sequence SEQ ID NO: 20 (referred to as anti-IgE-BCR-CAR, Q-CAR, or “Q”). In addition, these experiments were performed to exclude the possibility of Q-CAR interaction with free IgE.

In the first experiment, high amounts of free human IgE (5 μg/mL equivalent to 2000 IU/mL) were added to the co-culture media of Q-CAR-T cells (anti-M1′-CAR-T or Q-CAR T-cells) with a population of NALM6 cells or NALM6 expressing long-IgE-BCR-surrogate construct (NALM6-L) CAR-T transduction rate was approximately 50%. Untransduced T cells were used as a control. E:T was 1:2 and co-culture was for 8 hours; experiments were performed in triplicate (n=3). As is shown in FIG. 11, the Q-CAR T cells' activation was not affected (as measured by IFN-gamma production), as was expected of its design to target M1′ that is specific to IgE in the form of BCR only.

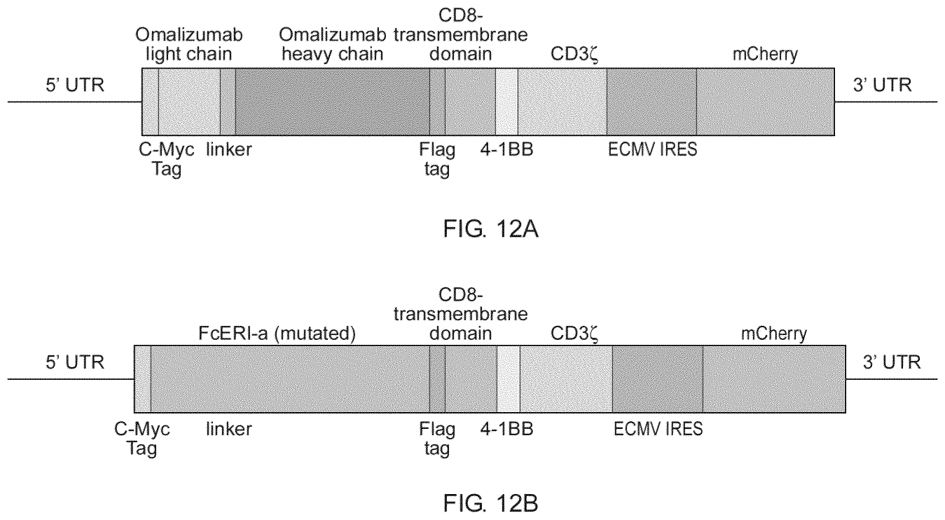

Next, the hypothesis that CAR against IgE, that is not BCR-specific and targets a domain that is part of secreted IgE as well, will be engaged by free IgE, which is abundant in the blood of atopic patients, and might lead to massive continuous activation of the CAR-T or prevent the CAR-T cell from reaching and killing its target (i.e. IgE-class switched B cells), or both, was tested. To test this hypothesis, two additional CARs were designed based on designs of previously published CAR-T anti-IgE, both targeting the IgE-Cε3 domain, and are identical to Q-CAR in all but the ectodomain: the scFv of the first design was adopted from Omalizumab (therapeutic anti IgE antibody) sequence coding for the variable domains (henceforth referred to as “O-CAR” or “O”) (FIG. 12A); The ectodomain of the second CAR is a mutated FcεRIα, the alfa sub unit of the high affinity IgE-Receptor (henceforth referred to as “F-CAR” or “F”) (FIG. 12B). The latter design is similar (but not identical) to an anti-IgE CAR design published in Ward D E et al. Chimeric Antigen Receptors Based on Low Affinity Mutants of FcϵRI Re-direct T Cell Specificity to Cells Expressing Membrane IgE. Front Immunol 2018; 9:2231.

In the following experiments the transduction rates of T cells with each of three CAR was similar in the range of ˜50-80%; the rate of target cells NALM6 expressing membrane-IgE like structures was ˜50%.

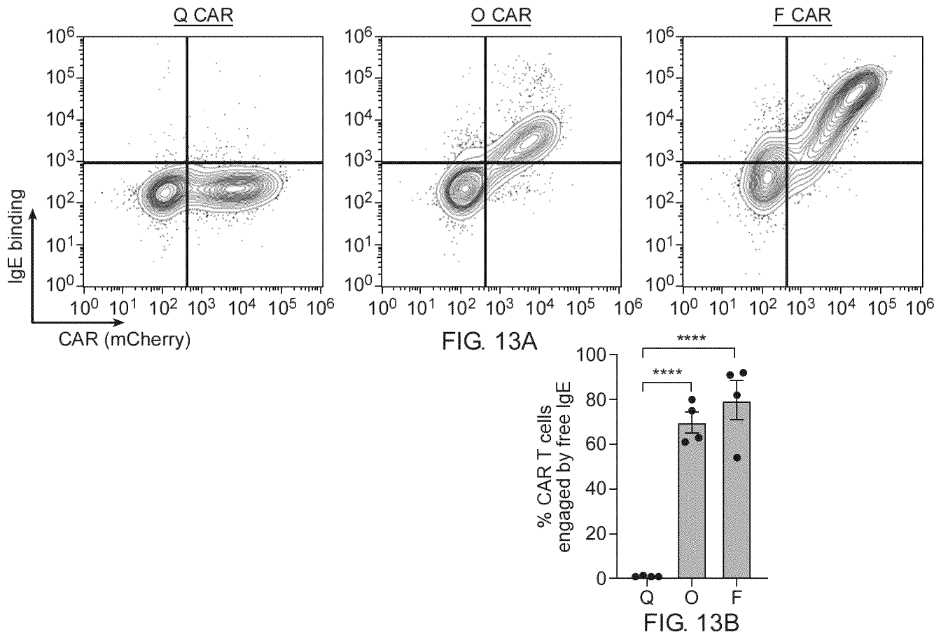

At first, the CAR-T cells or non-transduced T cells were cultured alone in medium supplemented by free IgE and measured IFNg production. Already upon a very short-term incubation of 15 minutes of CAR-T cells in medium supplemented with high free IgE of 5 μg/mL, it was evident that O-CAR T cells and F-CAR-T cells interact with and are engaged by IgE, while Q-CAR T cells are not. Staining of CAR T cells following incubation with free IgE was performed as follows: 250,000 T cells or related CAR T cells (Q, O, and F in parallel), were cultured in a 96-well plate for a short-term incubation of 15 minutes in MEM-alfa culture medium supplemented by free human IgE 5 μg/mL. Next, cells were collected, centrifuged at 500 g for 5 minutes, washed 2 times with PBS, and then stained with an anti-IgE antibody (BV) and anti-CD3 (FITC). Fluorescence intensity was assessed, for the two mentioned fluorophores and for mCherry expressed in transduced cells, using an Attune NxT Flow Cytometer. Negative controls included isotype antibodies and untransduced T cells stained with the test antibodies. As shown in FIG. 13A flow cytometry analysis of Q, O, and F anti-IgE CAR-T cells, after 15 min incubation with free human IgE 5 μg/mL and staining with anti-CD3 and anti-IgE, demonstrates that free IgE is engaged on O and F CARs but not Q CAR. Shown in FIG. 13B is a graphic representation of FIG. 13A, and results are presented as the mean percent of IgE-engaged-CARs of four different experiments.

Next, the various CAR-T constructs were incubated for 8 hours at 37° C., with and without human IgE 5 μg/mL supplemented in medium, and activation of the CAR-T cells was assessed by measuring IFN-gamma production (using ELISA) in the supernatant. Cells were plated in triplicates in a 96-well plate, 100K CAR-T cells/well in 200 microliters RPMI medium with 10% FBS 1% PS antibiotics, 25 IU rhIL2/mL. n=3. As shown in FIG. 14, O- and F-CAR-T cells were activated upon addition of free IgE to culture media, while Q-CAR T cells activation was not affected.

To assess the effect of IgE supplement on killing via Luciferase killing assay, the following experiment was performed:

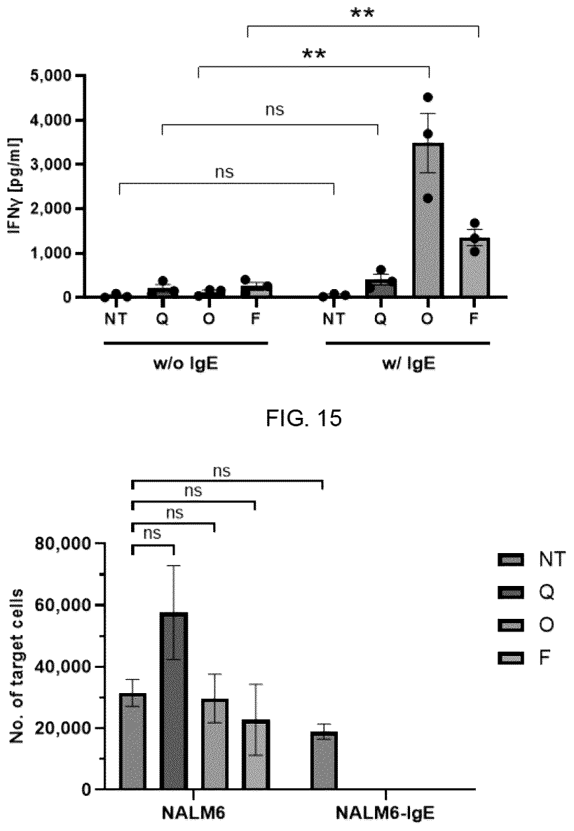

First, to show that O and F have a comparable killing ability to Q a co-culture experiment was performed in a 96-well plate, in technical duplicates, 200K/well anti-IgE-CAR T cells Q/O/F were co-cultured for 13 h E:T 2:1 with NALM6(Luc+) or NALM6-L(Luc+) in 200 microliter RPMI medium supplemented by 10% FBS 1% PS antibiotics. Control experiments were held in parallel with non-transduced T (activated, same donor). At the end of the co-culture, the plate was centrifuged at 500 g for 5 min, 100 microliters of supernatant were removed and replaced by Luciferin containing buffer (“ONE-Glo” Promega). The plate was read after 3 minutes and luminescence measured. As a standard scale, plates with an escalating measured number of cells were plated. As shown in FIG. 15, within 13 hours, all anti-IgE-CAR T cells completely eliminated the NALM6-L cells (here labelled NALM6-IgE), while elimination of NALM6 was similar to elimination by non-transduced T cells n=3.

In a following experiment, in a 96-well plate, in technical duplicates, 300K/well anti-IgE-CAR T cells (Q/O/F) were co-cultured for 13 h E:T 4:1 with NALM6-L(Luc+) (here labelled NALM6-IgE) in 250 microliter of RPMI medium supplemented by 10% FBS 1% PS antibiotics, either with or without IgE supplement 5 micrograms/mL. Control experiments were held in parallel with non-transduced T (activated, same donor). n=3. At the end of the co-culture, the plate was centrifuged at 500 g for 5 min, 100 microliters of supernatant were removed and replaced by 150 microliters of Luciferin-containing buffer (“ONE-Glo” Promega). The plate was read after 3 minutes and luminescence measured. As a standard scale, plates with an escalating measured number of cells were plated. As shown in FIG. 16, the killing ability of O and F CAR-T cells was reduced significantly by the addition of free IgE to the culture media, while Q-CAR T cells were not affected, nor were non-transduced T cells.

### In Conclusion:

Q-CAR T cells eliminate NALM6 cells expressing Long-IgE-BCR constructs, and killing is not affected by high free IgE in the culture, whereas O and F CAR-T cells, which are not specific to the IgE in its BCR isoform, are affected by free IgE and result in reduced killing ability.

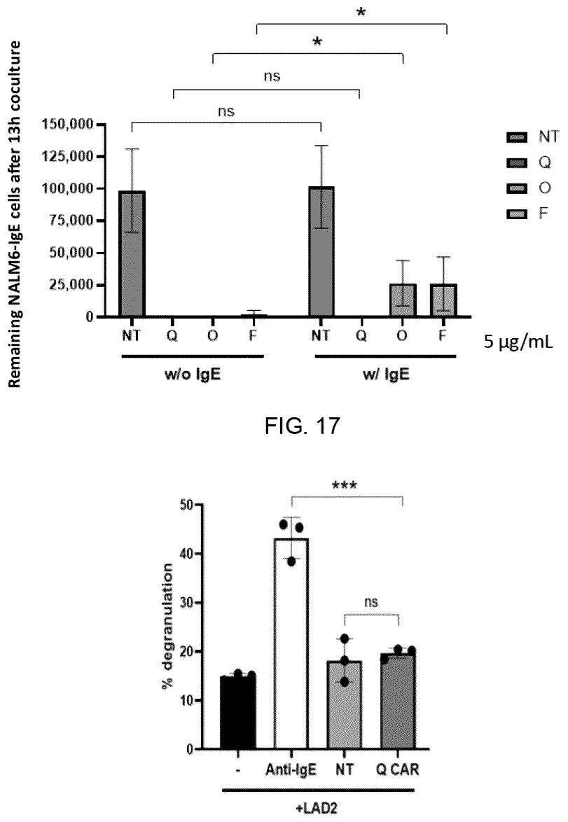

To exclude the possibility of Mast cell and basophil degranulation from an inadvertent interaction of the Q-CAR T with IgE while bound to its high affinity receptor FcϵRI, co-culture experiments were performed of Q-CAR T cells with LAD2 immortalized mast cells that were pre-incubated with human IgE. LAD2 Mast cells were pre-incubated with human IgE 1 μg/ml and then co-cultured with activated T cells or Q-CAR T cells (1:1 E:F ratio, 50% CAR transduction rate among Q-CAR T cell population). The hexosaminidase-β activity was determined using PNAG as substrate, and percentage of degranulation was calculated. Experimental details are more fully described in the Methods section hereinabove. The results of the assay are presented in FIG. 17. The data is presented as mean±SD (3 different donors). Statistics: ns=non-significant, *p<0.05, **p<0.01, ***p<0.001, ****p<0.0001

Q-CAR T cell incubation with LAD2 cells did not lead to mast cells' degranulation, while the positive control of anti-human IgE antibodies did.

### Example 3

### Anti-IgE-BCR CAR Specifically Abolishes IgE Production/Secretion by Human B Lymphocytes but not IgG Production/Secretion

In this experiment, levels of secreted IgE and IgG were measured in the supernatant of a co-culture of human B lymphocytes and donor T-lymphocytes transduced with Q-CAR (“Q”).

Human mononuclear cells were extracted from human tonsils and stored frozen in liquid nitrogen. Vials were thawed and B lymphocytes were isolated using a Human B isolation kit (Stemcell) and cultured using the “Immunocult” human B Cell expansion kit (Stemcell), 1 million cells/mL.

T lymphocytes from the same donor were activated and transduced with a retroviral vector coding for the Q CAR.

Co-culture of Effectors (activated T cells and CAR-T cells) and B cells was performed 2-10 days post-transduction of the donor T cells. Effectors and B cells were co-cultured for two days, in RPMI medium supplemented with 200 IU/mL rhIL-4, and 1 μg/mL anti-CD40 antibody (clone G28.5), with an T:B ratio of 1:2, and B cells at concentration of 500,000/mL, each combination was performed in triplicate; n=3. After 48 h, IgE and IgG in the supernatant of co-culture wells were measured using ELISA after removal of cells. Briefly, IgE ELISA was performed as follows: 96-well high binding microplates (Greiner Bio-One) were pre coated with capture antibody (for IgE anti h-IgE Mabtech mAbs107 concentration 2 μg/mL PBS, 100 μL/well, for IgG G protein 2 μg/mL PBS, 25 μL/well), the day after wells were “blocked” for one hour: for IgE-blocking by PBS with 1% FBS 200 μL/well, for IgG-blocking by 3 mg % skimmed bovine milk in PBS 150 μL/well. then plates were thoroughly washed 3× in Tween20 0.05% in PBS.

For IgE quantification, samples were diluted 1:2 fold and a standard was made using purified human IgE serially diluted in PBS. For IgG quantification, samples were diluted 1:200, a standard was made using purified human IgG serially diluted. Samples and standards were incubated for two hours. Plates were then washed 3× with Tween20 0.05%. For IgE detection, plates were then applied with biotin-conjugated anti human IgE (Mabtech mAbs 107/182/101) 2 μg/mL PBS+0.1% BSA, 100 μL/well, for one hour incubation, followed by wash ×3 with Tween20 0.05%, and application of Streptavidin-HRP 100 μL/well for 1 hour incubation. For IgG detection, anti-human IgG conjugated to HRP (Peroxidase AffiniPure™ Goat Anti-Human IgG (H+L) Jackson ImmunoResearch) 1:5000 (in milk 3%+Tween20 0.5%) 25 μL/well and were incubated for 30 minutes. This step was also followed by ×3 wash.

Finally, TMB substrate 100 μL/well (25 μL/well for IgG ELISA) was added and plate kept in the dark, and enzymatic activity quenched upon addition of H2SO4 2N 100 μL/well (25 μL/well for IgG ELISA). Detection was done in a Synergy M1 Plate reader (Biotek) (at wave length 450 nm minus background at wave length 540 nm). The concentration was determined by reference to the dilution factor of the standard curve.

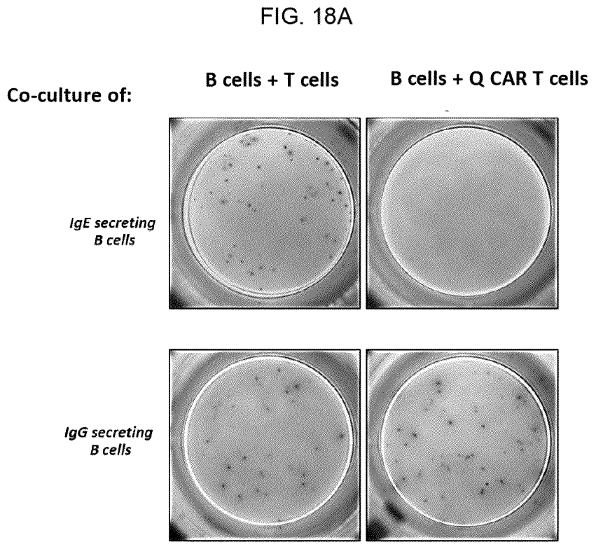

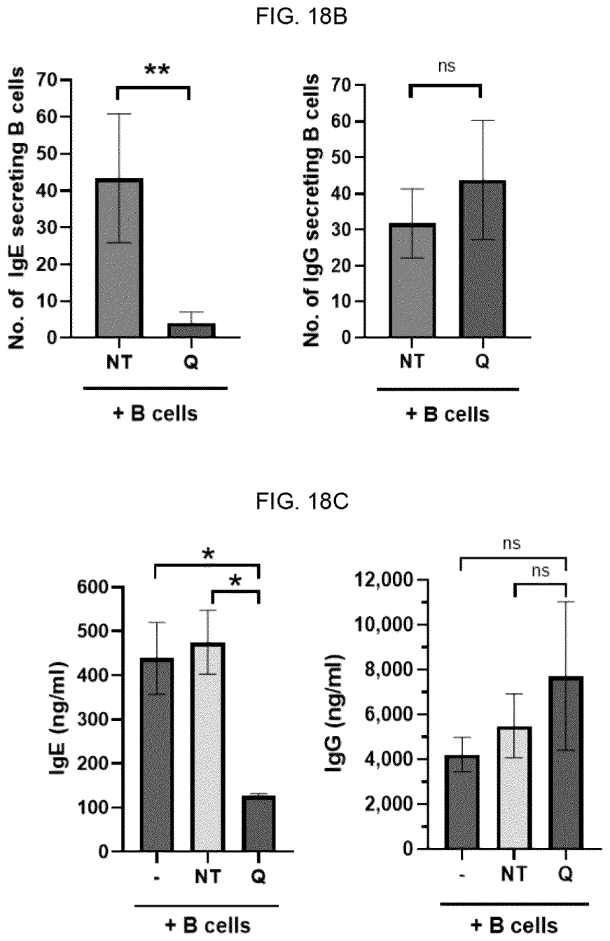

Results from the experiment conducted with 3 different donors are shown in FIGS. 18A and 18B. The results demonstrate that Q-CAR T cells significantly diminishes IgE production, but not IgG production, in co-culture of human tonsillar lymphocytes. While co-culture of B cells with Q-CAR T cells (“Q”) led to a significantly decreased IgE production, in comparison to non-transduced T cells, IgG production was not decreased. This points to a specific effect on IgE-BCR bearing cells.

### Example 4

### Anti IgE-BCR CAR Specifically Eliminated IgE Producing B Cells but not IgG-Producing B Cells

Human mononuclear cells were extracted from blood and human tonsils and stored frozen in liquid nitrogen. B lymphocytes from thawed vials were isolated using Human B isolation kit, (Stemcell) and cultured in Immunocult (Stemcell) human B Cell expansion kit, 1 million/mL.

T lymphocytes from the same donor were activated and transduced with anti-IgE-BCR CAR, as discussed before. 2-10 days post transduction Effectors (activated T cells and CAR-T cells) and B cells were co-cultured for two days, in RPMI medium supplemented with rhIL-4 200 IU/mL and anti-CD40 antibody 1 microgram/mL (clone G28.5), T:B 5:1, B cells at concentration of 150,000/mL. After 48 hours, cells were washed twice and transferred with fresh same medium to an ELIspot plate pre-coated with anti-IgE antibodies 15 microgram/mL.

ELIspot plates coated with IgE antibodies were prepared as follows. Prior to experiment, ELIspot Immobilon P membrane plates (Millipore) were pre-washed with ethanol 35% 20 μL/well, followed by wash with DDW 5× times and then coated with anti-IgE antibodies 15 μg/mL PBS (Mabtech mAbs107), 100 μL/well for overnight incubation. Later, ELIspot plates underwent blocking with RPMI medium with 10% FBS 200 μL/well for 30 minutes, and emptied before transfer of co-cultured cells.

After 20-24 hours of co-culturing at 37° C. in a 5% CO2 incubator, ELIspot plates were washed 5× with PBS and detecting Anti-IgE antibodies conjugated to biotin were added (Mabtech mAbs 107/182/101) for one hour incubation. Plates were then washed 5× and later streptavidin-conjugated to HRP was added for 30 minutes incubation. Following the 5× wash, development was performed with an AEC Chromogen Kit (Sigma-Aldrich). Finally, plates were dried and analyzed by iSpot ELISPOT reader (AID). Spots formed on the bottom of the well represent IgE secreting cells.

For ELIspot detection of IgG secreting cells—the protocol differed in the following stages: ELIspot plates were coated with IgG protein 1 μg/mL; only ⅙ of the primary coculture well's cell content was transferred to the ELIspot well; for detection, anti-human IgG conjugated to HRP (Peroxidase AffiniPure™ Goat Anti-Human IgG (H+L) Jackson ImmunoResearch) was used Spots formed on the bottom of the well represent IgG excreting cells.

IgE secreting cells were detected following 72 h co-culture of human T and B cells, but these cells could barely be traced upon co culture of the same B cells with Q-CAR T cells, as can be seen on the ELIspot plates shown in FIG. 18A. As can further be seen in FIG. 18A, IgG producing cells were abundant and their number was not diminished by Q-CAR T cells. Shown in FIG. 18B is quantification of number of immunoglobulins IgE (left panel) and IgG (right panel) secreting cells as depicted by ELISpot, represented as a mean of three different donors. Statistics: ns=non-significant, *p<0.05, **p<0.01, ***p<0.001, ****p<0.0001.

In accordance with the ELIspot findings, IgE production as measured by ELISA specifically in the supernatant of co-culture of B cells and Q CAR-T cells after 48 h was significantly reduced while IgG production in all co-cultures was preserved (FIG. 18C).

### Example 5

### Q-CAR T Cells Halted In Vivo Tumor Expansion and Prolonged Survival in an NSG Mouse Model

The M1′ domain of IgE BCR is unique to humans and several non-human primates, while it is absent in other mammals including mice. Therefore, in order to test the activity of human Q-CAR T cells in vivo immunodeficient mice NOD.Cg-PrkdcscidIl2rgtm1Wjl/SzJl (“NSG™”) were used as a host and injected with the “NALM6-Long” cells that also express the firefly Luciferase enzyme (“NALM6-Luc-IgE”).

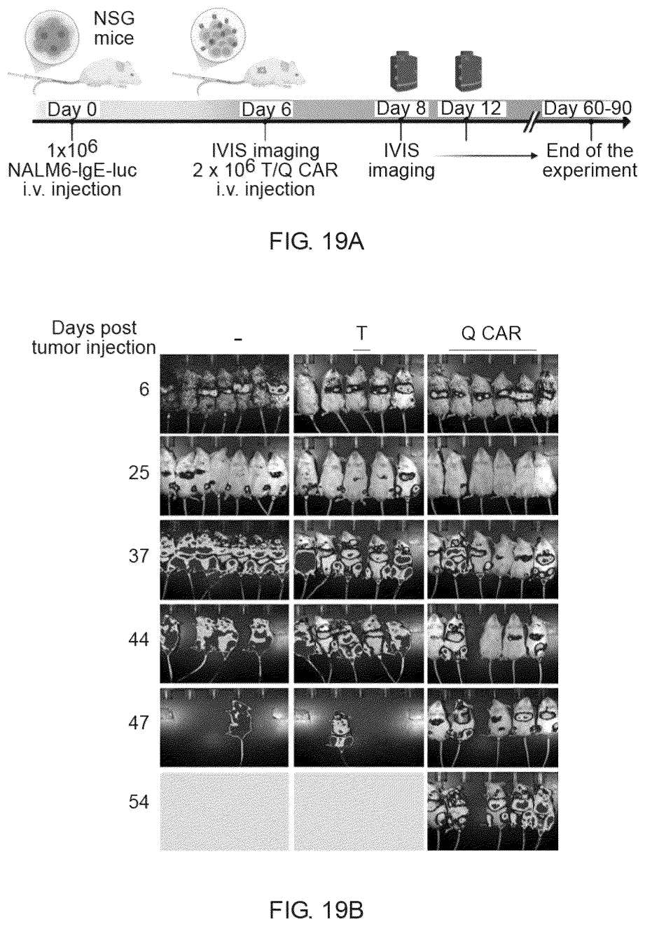

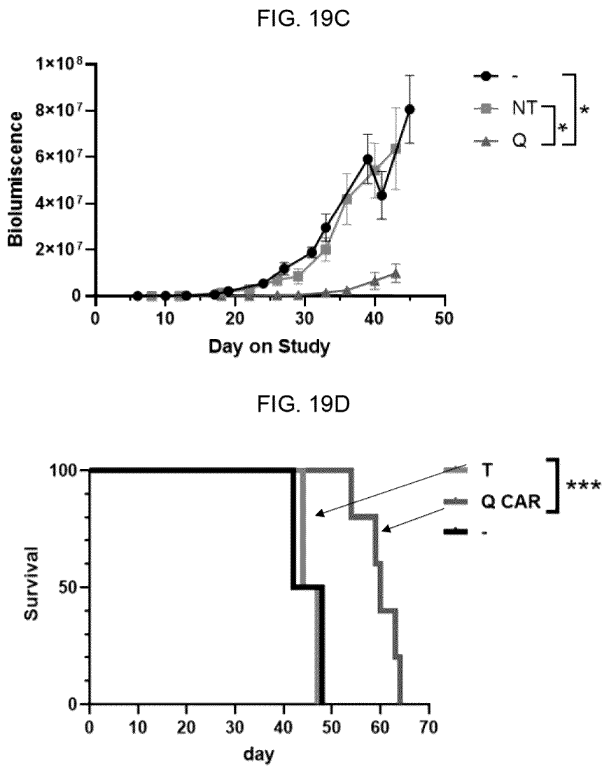

On day 0, 1 million NALM6-Luc-IgE cells were injected IV to tail vain. Upon visibility of the expending tumor (proliferation of the cancerous NALM6 cells) by IVIS imaging system (Xenogen), on day 6, 2 million Q-CAR-T cells (CAR positive in over 10% of them) were injected to six male mice. As a control group, 5 mice were injected with 2 million activated T cells from the same donor that did not undergo CAR retro transduction and 7 mice received no treatment. Mice were followed twice weekly, and bioluminescence levels, appearance and weight were recorded. The experimental scheme is shown in FIG. 19A. Further experimental details can be found in the Methods section hereinabove.

Neither T cells nor CAR-T cells could be detected in peripheral blood drawn a week after their injection; the next analysis of cell populations was performed upon euthanization (except mice that died spontaneously). Very soon after injection of the effector cells, significant differences in the rate of tumor expansion were detected between the groups that received Q CAR-T and the other two groups. Specifically, FIG. 19B shows a photo of the mice of the three groups on days 6, 25, 37, 44, 47, and 54 in which the group that received Q-CAR-T cells lived longer with significantly less tumor expansion. In addition, FIG. 19C shows the IVIS 1 min exposure graph, in which the group that received Q CAR-T had significantly less bioluminescence, demonstrating significant tumor expansion control by Q-CAR T cells in vivo.

Overall, Q-CAR-T cells treatment proved to be highly efficient in controlling tumor expansion, and prolonging survival, as shown in the Kaplan-Meier curve of FIG. 19D.

Although the present invention has been described herein above by way of preferred embodiments thereof, it can be modified, without departing from the spirit and nature of the subject invention as defined in the appended claims.

1. An immune cell engineered to express a chimeric antigen receptor (CAR), wherein the CAR comprises an antigen binding domain (ABD) comprising three complementarity-determining regions (CDR) of a light-chain variable domain (VL) having the amino acid sequence as set forth in SEQ ID NO: 12 and three CDR of a heavy-chain variable domain (VH) having the amino acid sequence as set forth in SEQ ID NO: 13, wherein the ABD binds specifically to an amino acid sequence of an extracellular domain of a human B-Cell-Receptor IgE (IgE-BCR).

2. The immune cell according to claim 1, wherein the ABD is characterized by at least one of:
(i) the ABD binds specifically to an amino acid sequence of the extracellular domain of a human IgE-BCR comprising the amino acid sequence set forth in SEQ ID NO: 1;
(ii) the ABD comprises a set of 6 complementarity-determining region (CDR) sequences, wherein the heavy chain CDR1 comprises the amino acid sequence SEQ ID NO: 6, the heavy chain CDR2 comprises the amino acid sequence SEQ ID NO: 7, the heavy chain CDR3 comprises the amino acid sequence SEQ ID NO: 8, the light chain CDR1 comprises the amino acid sequence SEQ ID NO:9, the light chain CDR2 comprises the amino acid sequence SEQ ID NO: 10, and the light chain CDR3 comprises the amino acid sequence SEQ ID NO: 11;
(iii) the ABD comprises a light-chain variable domain (VL) having the amino acid sequence as set forth in SEQ ID NO: 12 and a heavy-chain variable domain (VH) having the amino acid sequence as set forth in SEQ ID NO: 13; or
(iv) the ABD is a single chain variable fragment (scFv) comprising the amino acid sequence SEQ ID NO: 14.

3. The immune cell according to claim 1, wherein the CAR comprises a transmembrane domain (TM domain), a costimulatory domain, an activation domain and a leading peptide.

4. The immune cell according to claim 3, wherein the CAR is characterized by at least one of:
(i) the TM domain is a TM domain of a receptor selected from CD28 and CD8;
(ii) the costimulatory domain is selected from a costimulatory domain of a protein selected from CD28, 4-1BB, OX40, iCOS, CD27, CD80, CD70 and a combination thereof;
(iii) the TM domain and the costimulatory domain are both derived from CD28;
(iv) the antigen binding domain is linked to the TM domain via a spacer;
(v) the activation domain is selected from FcRγ and CD3-ζ activation domains; or
(vi) the TM domain is a TM domain of a receptor selected from CD28a, the costimulatory domain is a costimulatory domain of 4-1BB and the activation is CD3-ζ activation domain.

5. The immune cell according to claim 4, characterized by at least one of (i) the TM domain comprises the amino acid sequence SEQ ID NO: 16, (ii) the costimulatory domain comprises the amino acid sequence SEQ ID NO: 17, (iii) the activation domain comprises the amino acid sequence SEQ ID NO: 18, (iv) the leading peptide comprises the amino acid sequence SEQ ID NO: 19; or the CAR comprises the amino acid sequence SEQ ID NO: 20.

6. The immune cell according to claim 1, wherein the cell is characterized by at least one of:
(i) the immune cell is engineered to express the CAR upon a stimulus;
(ii) the immune cell is engineered to express an immune system effector;
(iii) the immune cell is selected from a T cell, a natural killer cell and a macrophage;
(iv) the immune cell is T cell selected from a CD4+ T cell and a CD8+ T cell.

7. A composition comprising a plurality of immune cells according claim 1, and a carrier.

8. The composition according to claim 7, wherein the composition is a pharmaceutical composition and the carrier is a pharmaceutically acceptable carrier.

9. The pharmaceutical composition of claim 8, comprising a plurality of the immune cells, wherein the immune cells are T cells.

10. A chimeric antigen receptor (CAR) comprising an antigen binding domain (ABD) comprising three complementarity-determining regions (CDR) of a light-chain variable domain (VL) having the amino acid sequence as set forth in SEQ ID NO: 12 and three CDR of a heavy-chain variable domain (VH) having the amino acid sequence as set forth in SEQ ID NO: 13.

11. The CAR according to claim 10, wherein the ABD is characterized by at least one of:
(i) the ABD binds specifically to an amino acid sequence of the extracellular domain of a human IgE-BCR comprising the amino acid sequence SEQ ID NO: 1;
(ii) the ABD comprises a set of 6 complementarity-determining region (CDR) sequences, wherein the heavy chain CDR1 comprises the amino acid sequence SEQ ID NO: 6, the heavy chain CDR2 comprises the amino acid sequence SEQ ID NO: 7, the heavy chain CDR3 comprises the amino acid sequence SEQ ID NO: 8, the light chain CDR1 comprises the amino acid sequence SEQ ID NO:9, the light chain CDR2 comprises the amino acid sequence SEQ ID NO: 10, and the light chain CDR3 comprises the amino acid sequence SEQ ID NO: 11;
(iii) the ABD comprises a light-chain variable domain (VL) having the amino acid sequence as set forth in SEQ ID NO: 12 and a heavy-chain variable domain (VH) having the amino acid sequence as set forth in SEQ ID NO: 13; or
(iv) the ABD is a single chain variable fragment (scFv) comprising the amino acid sequence SEQ ID NO: 14.

12. The CAR cell according to claim 11, wherein the CAR comprises a transmembrane domain (TM domain), a costimulatory domain, an activation domain and a leading peptide.

13. The CAR according to claim 12, wherein the CAR is characterized by at least one of (i) the TM domain is a TM domain of a receptor selected from CD28 and CD8; (ii) the costimulatory domain is selected from a costimulatory domain of a protein selected from CD28, 4-1BB, OX40, iCOS, CD27, CD80, CD70, and any combination thereof; (iii) the antigen binding domain is linked to the TM domain via a spacer; and (iv) the activation domain is selected from FcRγ and CD3-ζ activation domains.

14. The CAR according to claim 13, wherein the TM domain comprises the amino acid sequence SEQ ID NO: 16, the costimulatory domain comprises the amino acid sequence SEQ ID NO: 17, the activation domain comprises the amino acid sequence SEQ ID NO: 18 and the leading peptide comprises the amino acid sequence SEQ ID NO: 19.

15. The CAR according to claim 14, wherein the CAR comprises the amino acid sequence SEQ ID NO: 20.

16. A nucleic acid molecule encoding the CAR according to claim 10.

17. The nucleic acid molecule according to claim 16, comprising the nucleic acid sequence selected from SEQ ID NO: 21, 22, 23, 25, 26, 27, 28, a combination thereof, and SEQ ID NO: 29.

18. A nucleic acid construct or a vector comprising the nucleic acid molecule according to claim 16, wherein the vector further comprises a promoter operably linked to said nucleic acid.

19. A cell comprising the CAR according to claim 10, or a nucleic acid molecule encoding said CAR or a nucleic acid construct or the vector comprising nucleic acid molecule encoding said CAR.

20. A method for treating allergy in a subject in need thereof comprising administering to said subject a therapeutically effective amount of immune cells according to claim 1, or a pharmaceutical composition comprising the immune cells.
